SEQUENCE LISTING (16 sequences)

SEQ ID NO: 1 - protein, 264 aa, mouse
MLLAWVHTFLLSNMLLAEAYGSGGCFWDNGHLYREDQPSPAPGLRCLNWLAAQGSRESLTEPSPGNHNYCRNPDQDPRGPWCYISSETGVPEKRPCEDVSCPETTSQAPPPSSAMELEEKSGAPGDKEAQVFPPANALPARSEAAEVQPVIGISQLVRMNSKEKKDLGTLGYVLGITMMVIILAIGAGIIVGYTYKRGKDLKEQHEKKACEREMQRITLPLSAFTNPTCETVDENTIIVHSNQTPADVQEGSTLLTGQAGTPGA

SEQ ID NO: 2 - protein, 82 aa, mouse
GGCFWDNGHLYREDQPSPAPGLRCLNWLAAQGSRESLTEPSPGNHNYCRNPDQDPRGPWCYISSETGVPEKRPCEDVSCPET

SEQ ID NO: 3 - protein, 26 aa, mouse
LGTLGYVLGITMMVIILAIGAGIIVG

SEQ ID NO: 4 - protein, 23 aa, mouse
GKDLKEQHEKKACEREMQRITLP

SEQ ID NO: 5 - protein, 273 aa, Artificial Sequence
  synthetic construct
MALPVTALLLPLALLLHAARPDYKDDDDKIDGGCFWDNGHLYREDQPSPAPGLRCLNWLAAQGSRESLTEPSPGNHNYCRNPDQDPRGPWCYISSETGVPEKRPCEDVSCPETTSQAPPPSSAMELEEKSGAPGDKEAQVFPPANALPARSEAAEVQPVIGISQLVRMNSKEKKDLGTLGYVLGITMMVIILAIGAGIIVGYTYKRGKDLKEQHEKKACEREMQRITLPLSAFTNPTCETVDENTIIVHSNQTPADVQEGSTLLTGQAGTPGA

SEQ ID NO: 6 - protein, 21 aa, Artificial Sequence
  synthetic construct
MALPVTALLLPLALLLHAARP

SEQ ID NO: 7 - protein, 10 aa, Artificial Sequence
  synthetic construct
DYKDDDDKID

SEQ ID NO: 8 - protein, 82 aa, Artificial Sequence; identical to SEQ ID NO: 2
  synthetic construct

SEQ ID NO: 9 - protein, 26 aa, Artificial Sequence
  synthetic construct
LGTLGYVLGITMMVIILAIGAGIIVG

SEQ ID NO: 10 - protein, 23 aa, Artificial Sequence
  synthetic construct
GKDLKEQHEKKACEREMQRITLP

SEQ ID NO: 11 - protein, 286 aa, Artificial Sequence
  synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRSYTYKRGKDLKEQHEKKACEREMQRITLPLSAFTNPTCETVDENTIIVHSNQTPADVQEGSTLLTGQAGTPGA

SEQ ID NO: 12 - protein, 263 aa, Homo sapiens
MLLAWVQAFLVSNMLLAEAYGSGGCFWDNGHLYREDQTSPAPGLRCLNWLDAQSGLASAPVSGAGNHSYCRNPDEDPRGPWCYVSGEAGVPEKRPCEDLRCPETTSQALPAFTTEIQEASEGPGADEVQVFAPANALPARSEAAAVQPVIGISQRVRMNSKEKKDLGTLGYVLGITMMVIIIAIGAGIILGYSYKRGKDLKEQHDQKVCEREMQRITLPLSAFTNPTCEIVDEKTVVVHTSQTPVDPQEGSTPLMGQAGTPGA

SEQ ID NO: 13 - protein, 165 aa, Homo sapiens
MLLAWVQAFLVSNMLLAEAYGSGGCFWDNGHLYREDQTSPAPGLRCLNWLDAQSGLASAPVSGAGNHSYCRNPDEDPRGPWCYVSGEAGVPEKRPCEDLRCPETTSQALPAFTTEIQEASEGPGADEVQVFAPANALPARSEAAAVQPVIGISQRVRMNSKEKKD

SEQ ID NO: 14 - protein, 82 aa, Homo sapiens
GGCFWDNGHLYREDQTSPAPGLRCLNWLDAQSGLASAPVSGAGNHSYCRNPDEDPRGPWCYVSGEAGVPEKRPCEDLRCPET

SEQ ID NO: 15 - DNA, 22 bp, Artificial Sequence
  synthetic construct
atgctgttggctttgggtacac

SEQ ID NO: 16 - DNA, 19 bp, Artificial Sequence
  synthetic construct
cggcagtagttgtggttgc

(19) United States
(12) Patent Application Publication
(10) Pub. No.: US 2018/0250393 A1
(43) Pub. Date: Sep. 6, 2018

(54) MANIPULATION OF T CELL AND MAST CELL ACTIVATION BY PIK3IP1

(71) Applicant: UNIVERSITY OF PITTSBURGH-OF THE COMMONWEALTH SYSTEM OF HIGHER EDUCATION, Pittsburgh, PA (US)

(72) Inventors: Marie Colette DeFrances, Gibsonia, PA (US); Lawrence P. Kane, Pittsburgh, PA (US)

(21) Appl. No.: 15/909,322

(22) Filed: Mar. 1, 2018

Related U.S. Application Data
(60) Provisional application No. 62/465,376, filed on Mar. 1, 2017.

(51) Int. Cl. A61K 39/395; C07K 14/47; A61P 37/08; A61P 29/00

(52) U.S. Cl. CPC A61K 39/3955; C07K 14/4703; A61K 2039/505; A61P 29/00; A61P 37/08

(57) ABSTRACT

The present disclosure relates to compounds and compositions useful as PIK3IP1 agonists and methods of treating inflammatory diseases or allergic conditions or diseases.

### CROSS REFERENCE TO RELATED APPLICATIONS

This application claims the benefit of U.S. Provisional Patent Application Ser. No. 62/465,376 filed Mar. 1, 2017, the disclosure of which is expressly incorporated herein by reference.

### STATEMENT REGARDING FEDERALLY SPONSORED RESEARCH

This invention was made with government support under Grant No. AI126845 and AI095730 awarded by the National Institutes of Health. The Government has certain rights in the invention.

### FIELD

The present disclosure relates to compounds and compositions useful as PIK3IP1 agonists and methods of treating inflammatory diseases or allergic conditions or diseases.

### BACKGROUND

PI-3 kinases (PI3K) are a family of lipid kinases that are activated by numerous cellular receptors. Proper regulation of PI3K and its products is critical to cellular homeostasis. Activating mutations or amplification of PI3K and the downstream effector Akt have been implicated in various cancers. Conversely, at least two negative regulators of signaling downstream of PI3K function as tumor suppressors: the lipid phosphatases PTEN, and INPP4B. Downstream effectors of PI3K include many PH domain-containing proteins. Of particular relevance for antigen-responsive leukocytes like T cells and mast cells are the Tec family tyrosine kinases, which enhance PLC-γ1 activation, as well as the Akt family of serine/threonine kinases, which regulate cell survival, metabolism, growth and protein translation.

Recently another negative regulator of PI3K that acts more proximally to inhibit PI3K activity was described. PIK3IP1 (also known as Transmembrane Inhibitor of PI3K-TrIP) is a transmembrane protein that contains an extracellular kringle domain and a cytoplasmic domain with a region homologous to the inter-SH2 p110-binding domain of p85. PIK3IP1 can also function as a tumor suppressor, since transgenic expression of PIK3IP1 in the liver suppressed the development of hepatocellular carcinoma (HCC) in a susceptible mouse strain. What is needed are improved compounds that can regulate the PI3K pathway, in particular, by targeting the PIK3IP1 transmembrane protein.

The compounds, compositions, and methods disclosed herein address these and other needs.

### SUMMARY

Disclosed herein are compounds and compositions that are useful as PIK3IP1 agonists. In some embodiments, compounds that bind PIK3IP1 (for example, anti-PIK3IP1 antibodies) can be used in methods of treating or preventing an inflammatory disease or an allergic condition or disease.

In one aspect, disclosed herein is an isolated antibody or antigen-binding fragment thereof that specifically binds to a PIK3IP1 polypeptide, wherein the antibody or antigen-binding fragment thereof reduces the activity of T cells involved in mediating an inflammatory disease.

In another aspect, disclosed herein is an isolated antibody or antigen-binding fragment thereof that specifically binds to a PIK3IP1 polypeptide, wherein the antibody or antigen-binding fragment thereof reduces the activity of mast cells involved in mediating an allergic condition or disease.

In some embodiments, the antibody binds to an extracellular domain of PIK3IP1. In some embodiments, the antibody binds to a kringle domain of PIK3IP1. In some embodiments, the antibody is a monoclonal antibody.

In one embodiment, disclosed herein is a pharmaceutical composition comprising: an antibody or antigen-binding fragment thereof that specifically binds to a PIK3IP1 polypeptide; and a pharmaceutically acceptable excipient.

In one aspect, disclosed herein is a method for treating or preventing an inflammatory disease mediated by T cells, comprising administering to a subject in need thereof an effective amount of an antibody or antigen-binding fragment thereof that specifically binds to a PIK3IP1 polypeptide, wherein the antibody or antigen-binding fragment thereof reduces the activity of T cells involved in mediating the inflammatory disease. In some embodiments, the inflammatory disease is selected from diabetes mellitus, multiple sclerosis, or systemic lupus erythematosus.

In another aspect, disclosed herein is a method for treating or preventing an allergic condition or disease mediated by mast cells, comprising administering to a subject in need thereof an effective amount of an antibody or antigen-binding fragment thereof that specifically binds to a PIK3IP1 polypeptide, wherein the antibody or antigen-binding fragment thereof reduces the activity of mast cells involved in mediating the allergic condition or disease.

### BRIEF DESCRIPTION OF THE DRAWINGS

The accompanying figures, which are incorporated in and constitute a part of this specification, illustrate several aspects described below.

FIG. 1 shows that the kringle and cytoplasmic domains are required for PIK3IP1/TrIP activity. (A) Domain structure of TrIP, indicating the kringle, transmembrane and p85-like domains. (B) Expression of Flag-tagged WT TrIP on transiently transfected D10 T cells. (C) Flow cytometric analysis of pS6 activation in control (vector) or WT TrIP-transfected D10 T cells, stimulated with anti-CD3/CD28 for the indicated times. (D) Quantitation of pS6 activation as shown in panel C. (E) Expression of WT and mutant Flag-tagged TrIP constructs on transfected D10 cells. (F) Representative histogram of pS6 staining in D10 cells transiently transfected with Flag-tagged TrIP constructs and activated with anti-CD3/CD28. (G) Quantitation of pS6 activation obtained from flow cytometric analysis of activated D10 cells transfected with Flag-tagged TrIP constructs, as shown in panel F. Data in each panel are representative of at least three experiments.

FIG. 2 shows that cell-surface TrIP is downregulated during T cell activation. D10 cells transfected with WT TrIP were mixed with CH27 B cells as APCs (+/− pre-loading with conalbumin antigen) for the given time points. (A) Representative flow cytometry analyzing cell-surface expression of Flag-tagged WT TrIP on D10 cells stimulated with CH27 B cells alone (top row) or plus antigen (bottom row). (B) Quantitation of data shown in panel A. (C) Representative flow cytometry analyzing pS6 staining in D10 cells transfected with Flag-tagged WT TrIP and stimulated with CH27 B cells alone (top row) or plus antigen (bottom row). (D) Quantitation of data shown in panel C. Data are representative of three experiments.

FIG. 3 shows the kringle domain regulates TrIP expression and function. D10 cells transiently transfected with Flag-tagged WT or Δkringle TrIP were mixed with CH27 B cells plus antigen for the given time points. (A) Representative flow cytometry analyzing cell-surface expression of Flag-tagged WT (top row) or Δkringle (bottom row) TrIP on D10 cells stimulated with CH27 B cells plus antigen. (B) Quantitation of data shown in panel A. (C) Representative flow cytometry analyzing pS6 staining in the same cells as in panel A. (D) Quantitation of data shown in panel C. Data are representative of three experiments.

FIG. 4 shows that artificial dimerization of TrIP induces inhibition of T cell activation. (A) Structure of ecto hCD8-mPIK3IP cytoplasmic chimera, and its expression on transfected D10 cells. (B) Luciferase assay on D10 cells transiently transfected with Flag-tagged TrIP, hCD8-TrIP and luciferase constructs and activated with anti-CD3/CD28. (C) Flow cytometric analysis of pS6 after stimulation of hCD8-TrIP transiently transfected D10 cells in the presence of 10 μg/ml anti-hCD8. (D) Quantification of pS6 after stimulation of hCD8-TrIP transfected D10 cells in the presence of varying concentrations of anti-hCD8. Data are representative of three experiments.

FIG. 5 shows TCR signaling and TrIP function. D10 cells were transfected with control, WT TrIP or Δcyto-TrIP and stimulated with anti-CD3/CD28. (A) Representative flow cytometric analysis of Flag-tagged TrIP and pS6 expression of cells transfected with WT TrIP (top row) or Δcyto-TrIP (bottom row). (B) Quantitation of Flag expression over the course of stimulation; quantitation of total pS6⁺ cells at different time points. (C) TrIP surface expression (left) and pS6 staining (right) of cells expressing WT, Δ.kringle or Δ.p85-like TrIP, after stimulation with anti-CD3/CD28, as in panels A-B. (D) The cytoplasmic p85-like domain of TrIP binds to p110δ. 293 cells were transiently transfected with Flag-tagged WT TrIP and p85Δ-TrIP along with HA-tagged p110δ, as indicated. Co-immunoprecipitation and western blot analysis of TrIP and p110δ (top). Immunoprecipitated (IP) fractions showing Flag-tagged (TrIP) protein expression (middle). Whole cell lysate (WCL) showing HA-tagged (p110δ) protein expression (bottom). Data are representative of three experiments.

FIG. 6 shows that conditional TrIP KO mice exhibit higher TCR signaling. Splenocytes and lymphocytes were isolated from WT and KO mice and stimulated with anti-CD3/CD28 for the indicated times. (A) Flow cytometric analysis of pS6 expression on stimulated CD4⁺ cells. (B) Quantitation of data shown in panel A. (C) Flow cytometric analysis of pS6 expression on stimulated CD8⁺ cells. (D) Quantitation of data shown in panel C. (E-F) T cells were purified from spleen and lymph nodes of WT or TrIP KO mice and stimulated with anti-CD3/CD28 antibodies for the indicated times. Lysates were analyzed by western blot for phospho-Akt (T308-left; S473-right) and β-actin as a loading control. p values were calculated using two-way ANOVA with Sidak's multiple comparisons test. p values are represented with the following symbols: *0.01-0.05, **0.001-0.01, ***<0.001. Data in each panel are representative of at least three experiments.

FIG. 7 shows that TrIP KO T cells display a higher tendency for Th1 differentiation. Naïve T cells from WT and KO mice were cultured under neutral, Th0, Th1, Th17 and iTreg conditions for three days, re-stimulated and analyzed for (A) IFNγ and T-bet expression, (B) IL-17a and RORγt expression, and (C-D) Foxp3 and CD25 expression. (E) Naïve CD4⁺ T cells from the indicated mice were stimulated under the indicated conditions, RNA extracted and analyzed by qPCR for Plk3ip1 message. Data shown are normalized to naïve WT T cells. p values were calculated using two-way ANOVA with Sidak's multiple comparisons test. p values are represented with the following symbols: *0.01-0.05, **0.001-0.01, ***<0.001. Data are representative of more than three experiments.

FIG. 8 shows that TrIP KO CD4⁺ T cell phenotype is reversed by PI3K/Akt pathway inhibitors. (A) Naïve T cells from WT and KO mice were cultured under Th1 conditions for three days in the presence of PI3K inhibitors (Akti, LY294002 and IC-87114), re-stimulated with PMA/Ionomycin and analyzed for IFNγ and CD25 expression. (B) Quantitation of A. p values were calculated using two-way ANOVA with Sidak's multiple comparisons test. p values are represented with the following symbols: *0.01-0.05, **0.001-0.01, ***<0.001. Data are representative of more than three experiments.

FIG. 9 shows that TrIP KO mice are less susceptible to Listeria infection. (A) Liver extracts from WT (CD4-Cre only) and TrIP KO mice infected with LM-GP33 were obtained four days after infection and plated to measure bacterial burden. (B) Analysis of tetramer positive CD8⁺ effector T cells four days after infection. Plots represent CD44⁺ KLRG1⁺CD127⁻ (top) and CD44⁺ KLRG1⁻CD127⁺ (bottom) CD8⁺ T cells. (C) Model for the function of TrIP in T cells. Data are representative of two experiments with 4 mice per group.

FIG. 10 shows there are no obvious T cell developmental defects in CD4-cre conditional TrIP KO mice. (A) After confirmation of CD4-Cre and Pik3ip1 fox by tail snip genotyping, spleen and lymph node cells from naïve mice of indicated genotypes were screened by RT-PCR for the presence of Pik3ip1 mRNA. (B) Characterization of thymocytes and (C) splenocytes from WT, HET and KO mice.

FIG. 11 shows the specific binding of a mAb (clone H2) to TrIP-transfected cells. HEK293 cells were transfected with empty vector, WT mTrIP or a construct lacking the kringle domain (which constitutes most of the ecto portion). Cells were stained 2d later with anti-Flag (Y-axis) and the H2 clone.

FIG. 12 shows a model for negative regulation of PI3K by PIK3IP1. (Top) Alignment of p85β p110-interacting motif with the indicated cyto region of PIK3IP1 (ref (1)). (Bottom) PIK3IP1 may function to bind and sequester PI3K, preventing its activation at ligated receptors.

FIG. 13 shows the identification of mAb's to the ecto domain of mouse PIK3IP1. (L) HEK293 cells with or without Myc-tagged PIK3IP1 were stained via intracellular flow cytometry with Myc mAb 9B11. (R panels) The same 293 cells were stained with culture supernatants of the indicated anti-PIK3IP1 mAb clones.

FIG. 14 shows the binding of PIK3IP1 to Trem16. (L) HEK293 cells with or without Myc-tagged Trem16 were stained for intracellular flow cytometry with a-Myc mAb. (R) The same cells were stained with indicated amts. of PIK3IP1-Fc+2° Ab, or 2° Ab alone.

FIG. 15 shows grossly normal T cell development in the absence of PIK3IP1. CD4/CD8 profiles of lymph nodes from 4 week old WT or Sox2-Cre/PIK3IP1 KO mice. Total numbers of cells recovered were the same (not shown).

FIG. 16 shows enhanced TCR sensitivity of PIK3IP1 KO T cells. Splenic T cells from WT or Sox2-Cre/PIK3IP1 KO mice were stimulated for 24 hrs with anti-CD3 mAb, followed by flow cytometry analysis. The numbers indicate mean fluorescence intensity of WT (red) or KO (blue) T cells.

FIG. 17 shows the increased sensitivity of PIK3IP1 KO mast cells to IgE/antigen. Bone marrow cells were cultured for 5 wks in IL-3 containing media. BMMC were then stimulated o/n as indicated; secreted IL-6 was measured by ELISA.

FIG. 18 shows the normal differentiation of bone marrow mast cells (BMMC) from PIK3IP1 KO mice. Bone marrow cells were cultured for 5 wks in IL-3 containing media. BMMC were then analyzed by flow cytometry for c-kit and FcεRI.

### DETAILED DESCRIPTION

Disclosed herein are compounds and compositions that are useful as PIK3IP1 agonists. In some embodiments, compounds that bind PIK3IP1 (for example, anti-PIK3IP1 antibodies) can be used in methods of treating or preventing an inflammatory disease or an allergic condition or disease.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Terminology

As used in the specification and claims, the singular form “a,” “an,” and “the” include plural references unless the context clearly dictates otherwise. For example, the term “a cell” includes a plurality of cells, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, the term “mixture” can include solutions in which the components of the mixture are completely miscible, as well as suspensions and emulsions, in which the components of the mixture are not completely miscible.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

The terms “specific binding” or “specifically binding”, as used herein, in reference to the interaction of an antibody, a protein, or a peptide with a second chemical species, mean that the interaction is dependent upon the presence of a particular structure (e.g., an “antigenic determinant” or “epitope” as defined below) on the chemical species, for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains at least some portion of the epitope binding features of an Ig molecule (for example, allowing it to specifically bind to PIK3IP1). An antibody is said to be “capable of binding” a molecule if it is capable of specifically reacting with the molecule to thereby bind the molecule to the antibody.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

A “monoclonal antibody” as used herein is intended to refer to a preparation of antibody molecules, which share a common heavy chain and common light chain amino acid sequence, or any functional fragment, mutant, variant, or derivation thereof which retains at least the light chain epitope binding features of an Ig molecule, in contrast with “polyclonal” antibody preparations that contain a mixture of different antibodies. Monoclonal antibodies can be generated by several known technologies like phage, bacteria, yeast or ribosomal display, as well as classical methods exemplified by hybridoma-derived antibodies (e.g., an antibody secreted by a hybridoma prepared by hybridoma technology, such as the standard Kohler and Milstein hybridoma methodology ((1975) Nature 256:495-497).

The term “human PIK3IP1 protein”, as used herein refers to the protein encoded by SEQ ID NO:12, and variants thereof. In some embodiments, the human PIK3IP1 protein variant can be a naturally occurring variant. PIK3IP1 is also referred to herein as TrIP.

The term “mouse PIK3IP1 protein”, as used herein, refers to the protein encoded by SEQ ID NO:1, and variants thereof. In some embodiments, the mouse PIK3IP1 protein variant can be a naturally occurring variant. PIK3IP1 is also referred to herein as TrIP.

The term “variant” or “derivative” as used herein refers to an amino acid sequence derived from the amino acid sequence of the parent protein having one or more amino acid substitutions, insertions, and/or deletions.

### Compounds

Disclosed herein are compounds and compositions that are useful as PIK3IP1 agonists. In some embodiments, compounds that bind PIK3IP1 (for example, anti-PIK3IP1 antibodies) can be used in methods of treating or preventing an inflammatory disease or an allergic condition or disease.

In one aspect, disclosed herein is an isolated antibody or antigen-binding fragment thereof that specifically binds to a PIK3IP1 polypeptide, wherein the antibody or antigen-binding fragment thereof reduces the activity of T cells involved in mediating an inflammatory disease.

In another aspect, disclosed herein is an isolated antibody or antigen-binding fragment thereof that specifically binds to a PIK3IP1 polypeptide, wherein the antibody or antigen-binding fragment thereof reduces the activity of mast cells involved in mediating an allergic condition or disease.

In some embodiments, the antibody binds to an extracellular domain of PIK3IP1. In some embodiments, the antibody binds to a kringle domain of PIK3IP1. In some embodiments, the antibody is a monoclonal antibody. In some embodiments, the antibody is a humanized antibody.

In some embodiments, the PIK3IP1 agonist (for example, an anti-PIK3IP1 antibody) can interfere or block the binding of a PIK3IP1 binding protein or a PIK3IP1 ligand.

In some embodiments, the PIK3IP1 agonist can be a small molecule. In some embodiments, the PIK3IP1 agonist can be a small molecule, wherein the PIK3IP1 agonist binds the kringle domain of PIK3IP1. In some embodiments, the PIK3IP1 agonist can be a polypeptide.

In some embodiments, the PIK3IP1 protein is from a human. In some embodiments, the PIK3IP1 protein is from a mouse.

In some embodiments, the compound that binds PIK3IP1 is an antibody that binds to the human PIK3IP1 protein. In some embodiments, the antibody binds the amino acid sequence SEQ ID NO:12. In some embodiments, the compound that binds PIK3IP1 is an antibody that binds to the extracellular domain of the human PIK3IP1 protein. In some embodiments, the antibody binds the amino acid sequence SEQ ID NO:13. In some embodiments, the compound that binds PIK3IP1 is an antibody that binds to the kringle domain of the human PIK3IP1 protein. In some embodiments, the antibody binds the amino acid sequence SEQ ID NO:14. In some embodiments, the antibody or antigen-binding fragment thereof binds a fragment of a sequence selected from SEQ ID NO:12, SEQ ID NO:13, or SEQ ID NO:14.

In one embodiment, disclosed herein is an isolated antibody or antigen-binding fragment thereof that specifically binds to a PIK3IP1 polypeptide, wherein the antibody or antigen-binding fragment cross-links one or more PIK3IP1 polypeptides. In one embodiment, disclosed herein is an isolated antibody or antigen-binding fragment thereof that specifically binds to a PIK3IP1 polypeptide, wherein the antibody or antigen-binding fragment activates or increases the activity of the PIK3IP1 polypeptide.

In some embodiments, the antibody or antigen-binding fragment thereof is commercially available. For example, the anti-PIK3IP1 antibody can be selected from anti-PIK3IP1 antibody (ab87094) from Abcam, PIK3IP1 Antibody (PA5-43519) from Thermo Fisher, sc-365777 from Santa Cruz, DPATB-H81907 from Creative Diagnostics, or any other commercially available PIK3IP1 antibodies.

As used herein, the term “antibody or antigen-binding fragment thereof” encompasses chimeric antibodies and hybrid antibodies, with dual or multiple antigen or epitope specificities, and fragments, such as F(ab′)2, Fab′, Fab, Fv, sFv, and the like, including hybrid fragments. Thus, fragments of the antibodies that retain the ability to bind their specific antigens are provided. For example, fragments of antibodies which maintain PIK3IP1 binding activity are included within the meaning of the term “antibody or antigen-binding fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or antigen-binding fragment thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)).

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

Administration of the antibodies can be done as disclosed herein. Nucleic acid approaches for antibody delivery also exist. The antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### Compositions

Compositions, as described herein, comprising an active compound and an excipient of some sort may be useful in a variety of applications. For example, pharmaceutical compositions comprising an active compound and an excipient can be useful for the treatment or prevention of an inflammatory disease or an allergic condition or disease. In one embodiment, disclosed herein is a pharmaceutical composition comprising: an antibody or antigen-binding fragment thereof that specifically binds to a PIK3IP1 polypeptide; and a pharmaceutically acceptable excipient.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semi-solid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacilic acid/acrylamide copolymer, polyalkylene oxides such as polyethylene oxide, polypropylene oxide, poly(ethylene oxide-propylene oxide), and a Pluronic polymer, polyoxyethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Di stearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Di stearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxyalkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

### Methods of Treatment—Inflammatory Disease

The anti-PIK3IP1 antibodies and antigen binding agents described herein may be administered for the treatment of an inflammatory disease in a subject. Inflammation may arise as a response to an injury or abnormal stimulation caused by a physical, chemical, or biologic agent. An inflammation reaction may include the local reactions and resulting morphologic changes, destruction or removal of injurious material such as an infective organism, and responses that lead to repair and healing. The term “inflammatory” when used in reference to a disease or disorder refers to a pathological process, which is caused by, resulting from, or resulting in inflammation that is inappropriate or which does not resolve in the normal manner. Inflammatory diseases may be systemic or localized to particular tissues or organs.

In one aspect, disclosed herein is a method for treating or preventing an inflammatory disease mediated by T cells, comprising administering to a subject in need thereof an effective amount of an antibody or antigen-binding fragment thereof that specifically binds to a PIK3IP1 polypeptide, wherein the antibody or antigen-binding fragment thereof reduces the activity of T cells involved in mediating the inflammatory disease.

In one aspect, disclosed herein is a method for treating or preventing an inflammatory disease mediated by T cells, comprising administering to a subject in need thereof an effective amount of a PIK3IP1 agonist, wherein the antibody or antigen-binding fragment thereof reduces the activity of T cells involved in mediating the inflammatory disease.

In a further aspect, the disclosure relates to a method of treating or preventing an inflammatory disease, comprising administration of a therapeutically effective amount of a PIK3IP1 agonist (for example, an anti-PIK3IP1 antibody) to a subject in need thereof for the treatment of a disease selected from: diabetes mellitus, immune-mediated renal disease (glomerulonephritis, tubulointerstitial nephritis), demyelinating diseases of the central and peripheral nervous systems (such as multiple sclerosis), systemic lupus erythematosus, inflammatory bowel disease, rheumatoid arthritis, psoriatic arthritis, juvenile chronic arthritis, spondyloarthropathies, systemic sclerosis (scleroderma), idiopathic inflammatory myopathies (dermatomyositis, polymyositis), Sjögren's syndrome, systemic vaculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), idiopathic polyneuropathy, hepatobiliary diseases such as infectious hepatitis (hepatitis A, B, C, D, E and other nonhepatotropic viruses), autoimmune chronic active hepatitis, primary biliary cirrhosis, granulomatous hepatitis, and sclerosing cholangitis, inflammatory and fibrotic lung diseases (e.g., cystic fibrosis, eosinophilic pneumonias, idiopathic pulmonary fibrosis and hypersensitivity pneumonitis), gluten-sensitive enteropathy, Whipple's disease, autoimmune or immune-mediated skin diseases including bullous skin diseases, erythema multiforme and contact dermatitis, psoriasis, allergic diseases of the lung such as eosinophilic pneumonias, idiopathic pulmonary fibrosis and hypersensitivity pneumonitis, transplantation associated diseases including graft rejection and graft-versus host disease.

In one embodiment, the disclosure relates to a method of treating or preventing an inflammatory disease, comprising administration of a therapeutically effective amount of a PIK3IP1 agonist (for example, an anti-PIK3IP1 antibody) to a subject in need thereof for the treatment of a disease selected from: diabetes mellitus, multiple sclerosis, systemic lupus erythematosus, rheumatoid arthritis, psoriatic arthritis, or psoriasis.

### Methods of Treatment—Allergic Conditions or Diseases

In another aspect, disclosed herein is a method for treating or preventing an allergic condition or disease mediated by mast cells, comprising administering to a subject in need thereof an effective amount of an antibody or antigen-binding fragment thereof that specifically binds to a PIK3IP1 polypeptide, wherein the antibody or antigen-binding fragment thereof reduces the activity of mast cells involved in mediating the allergic condition or disease.

In one aspect, disclosed herein is a method for treating or preventing an allergic condition or disease mediated by mast cells, comprising administering to a subject in need thereof an effective amount of a PIK3IP1 agonist, wherein the antibody or antigen-binding fragment thereof reduces the activity of mast cells involved in mediating the allergic condition or disease.

Allergic reactions are generally immune reactions that are initiated by IgE-dependent stimulation of tissue mast cells and related effector molecules (e.g., basophils). Binding events between cell surface bound IgE molecules and antigen results in rapid release of biological response modifiers which bring about increased vascular permeability, vasodilation, smooth muscle contraction and local inflammation. This sequence of events is termed immediate hypersensitivity and begins rapidly, usually within minutes of exposure in a sensitized individual. In its most severe systemic form, anaphylaxis, such immediate hypersensitivity can bring about asphyxiation, produce cardiovascular collapse, and even result in death. Individuals that are prone to strong immediate hypersensitivity responses are referred to as “atopic”. Clinical manifestations of allergy or atopy include hay fever (rhinitis), asthma, urticaria (hives), skin irritation (e.g., eczema such as chronic eczema), anaphylaxis, and related conditions.

Furthermore, the present disclosure provides a novel method for treating atopic dermatitis, asthma, nasal inflammation, and other types of allergic disease. In some embodiments, the allergic condition or disease is selected from allergic atopic dermatitis, asthma, rhinitis, airway hyperresponsiveness, a food allergy, eosinophilic esophagitis, chronic urticaria, occupational allergy, allergic conjunctivitis, hay fever, airborne allergic sensitivities, stinging insect allergy, hypersensitivity pneumonitis, eosinophilic lung diseases, or drug allergies. In one embodiment, the allergic condition or disease is selected from allergic atopic dermatitis or asthma.

In one embodiment, disclosed herein is a method to treat an allergic condition or disease in a subject who has or is at risk of having an allergic condition or disease, comprising administering to the subject a composition comprising administering to a subject in need thereof an effective amount of an antibody or antigen-binding fragment thereof that specifically binds to a PIK3IP1 polypeptide.

### EXAMPLES

The following examples are set forth below to illustrate the compounds, compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. PIK3IP1/TrIP Restricts Activation of T Cells Through Inhibition of PI3K/Akt

Phosphatidylinositol-3 kinases (PI3Ks) modulate numerous cellular functions, including growth, proliferation and survival. Dysregulation of the PI3K pathway can lead to autoimmune disease and cancer. PIK3IP1 (or Transmembrane Inhibitor of PI3K-TrIP) is a novel transmembrane regulator of PI3K. TrIP contains an extracellular kringle domain and an intracellular “p85-like” domain with homology to the inter-SH2 domain of the regulatory subunit of PI3K. Although TrIP has been shown to bind to the p110 catalytic subunit of PI3K in fibroblasts, the mechanism by which TrIP functions is poorly understood. In this example, the kringle and “p85-like” domains are both shown as necessary for TrIP inhibition of PI3K. It was also demonstrated that TrIP protein is down-modulated from the surface of T cells to allow T cell activation. Using an inducible knockout mouse model, it was shown that TrIP-deficient T cells exhibit more robust T cell activation, show a preference for Th1 polarization and can mediate clearance of Listeria monocytogenes infection faster than WT mice. Thus, TrIP is an important negative regulator of T cell activation and is a novel target for immune modulation therapies.

### BACKGROUND

Phosphatidylinositide-3-Kinases (PI3K) are a family of lipid kinases that play important intracellular signaling roles in cellular processes such as proliferation, motility, growth, intracellular trafficking, differentiation and survival (Cantley, 2002; Fruman, 2007; Han et al., 2012). There are three main classes of PI3K. Class I PI3Ks, which are prevalent in immune cells, are composed of two subunits: a regulatory subunit (p85) and a catalytic subunit (p110) (Engelman, 2009; Fresno Vara et al., 2004; Fruman et al., 1998). During T cell receptor activation, PI3K is recruited to the plasma membrane via the SH2 domain of the p85 subunit. The associated p110 subunit is then activated to phosphorylate phosphatidylinositol 4,5-bisphosphate (PIP2) and produce phosphatidylinositol (3,4,5)-trisphosphate (PIP3). PIP3 interacts with the pleckstrin homology domain of Akt, causing a conformational change that allows PDK1 (kinase 3-phosphoinositide-dependent protein kinase-1) to partially activate Akt by phosphorylating threonine 308 (T308). Full activation of Akt is achieved by mTORC2-mediated phosphorylation at serine 473 (S473) and facilitates such processes as cell growth, cell cycle progression and cell survival. It is therefore not surprising that Akt amplification due to dysregulation of PI3K has been implicated in many cancers. In fact, several studies are presently focused on the development of PI3K pathway inhibitors as a means of combating various forms of cancer (Engelman, 2009).

Several negative regulators of PI3K have been identified (Agoulnik et al., 2011; Antignano et al., 2010; Carracedo and Pandolfi, 2008; Dillon and Miller, 2014). PTEN (phosphatase and tensin homologue deleted on chromosome 10) and SHIP-1 (SH2 containing inositol 5′-phosphatase) are phosphatases that dephosphorylate PIPS to PIP2, thereby inhibiting downstream signaling in the PI3K pathway. INPP4B (inositol polyphosphate 4-phosphatase type II) has been shown to dephosphorylate PIP2, thereby playing a role in the negative regulation of the PI3K pathway. Several studies have shown that loss of function mutations or deletions of these phosphatases can lead to dysregulated PI3K activity.

While the above phosphatases act downstream of PI3K, PIK3IP1 (PI3K-interacting protein-1, referred to as TrIP (transmembrane inhibitor of PI3K) for simplicity) is a recently identified inhibitor that acts upstream of the aforementioned phosphatases (DeFrances et al., 2012; Zhu et al., 2007). TrIP is a transmembrane protein composed of two main domains, an extracellular kringle domain and an intracellular tail that includes a motif similar to the p110-binding inter-SH2 domain found in the p85 subunit of PI3K. Overexpression of TrIP in mouse hepatocytes leads to a reduction in PI3K signaling and suppression of hepatocyte carcinoma development (He et al., 2008). Furthermore, recent work in cancer genetics highlights the transcriptional down-regulation of TrIP as a contributing factor to dysregulated PI3K signaling in tumorigenesis (Wong et al., 2014). Although it has been shown that TrIP inhibits PI3K by binding the p110 subunit via the p85-like domain, the role of the kringle domain remains to be determined. Given the ability of kringle domains in other proteins to bind to various ligands, it is possible that the TrIP kringle domain may bind one or more ligands for modulation of TrIP activity (Christen et al., 2010; Mikels et al., 2009; Patthy et al., 1984). Because TrIP is highly expressed in immune cells, particularly mast cells and T cells (DeFrances et al., 2012), it was investigated how the structure of TrIP enables regulation of PI3K in the context of the activated T cell.

In this example, the importance of both the kringle and p85-like domains to TrIP function in activated T cells was investigated. It was also examined how cell fate decisions and immune response are regulated by TrIP. In this example, it is shown that both the extracellular kringle domain and the intracellular p85-like domain are necessary for inhibition of PI3K by TrIP. Unexpectedly, it was also shown that cell-surface levels of TrIP are decreased upon T cell activation, which correlates with the upregulation of PI3K pathway signaling. Using a T cell conditional knockout mouse model, the loss of TrIP in T cells leads to an increase in T cell activation, which translates to stronger Th1 inflammatory potential and more rapid clearance of L. monocytogenes infection.

### Materials and Methods

### Cell Lines and Transfections

The D10 Th2 T cell clone (D10.G4.1; ATCC TIB-224) were maintained in RPMI media supplemented with 10% bovine growth serum (BGS), penicillin, streptomycin, glutamine and 25 U/ml recombinant human IL-2. Human embryonic kidney (HEK) 293 cells were maintained in DMEM media supplemented with 10% bovine growth serum (BGS), penicillin, streptomycin and glutamine. CH27 cells (mouse lymphoma; RRID:CVCL_7178) were maintained in RPMI media supplemented with 10% bovine growth serum (BGS), penicillin, streptomycin and glutamine. For structure-function assays, using a BIO-RAD GenePulser Xcell, D10 cells were individually electroporated with control plasmid, flag tagged wt-TrIP or flag tagged mutant TrIP constructs, along with pMaxGFP plasmid (encoding GFP from copepod Pontellina p.). One day after transfection, cells were evaluated by flow cytometry for GFP expression and TrIP expression (via anti-flag staining) using anti-DYKDDDDK APC clone L5 (BioLegend cat#637308). For p110δ interaction assays, flag-tagged TrIP variants and HA-tagged p110δ were transfected into HEK293 cells using TranslT-LT1 (Mirus) according to the manufacturers protocol. Cells were evaluated by western blot for expression using Roche anti-HA (clone 12CA5; Cat#11 583 816 001) and BioLegend Direct-Blot HRP anti-DYKDDDDK (Clone L5; cat#637311).

### T Cell/APC Co-Cultures

CH27 cells were pulsed with 100 μg/ml chicken conalbumin (Sigma C7786) one day before mixing with D10 cells. D10 cells were mixed in a 1:1 ratio with either pulsed unpulsed CH27 cells for 15, 30 and 60 mins. Reactions were quenched by placing cells on ice, adding 1 ml of PBS and centrifugation to settle cells and decant activation media. Cells were then stained with anti-DYKDDDDK APC clone L5 (BioLegend cat#637308), anti-mouse CD19 violetFluor 450 (clone 1D3; Tonbo Biosciences 75-0193-U025) and anti-mouse CD4 Brilliant Violet 510 (clone GK1.5; BioLegend #100449). Cells were washed three times in PBS and then fixed and permeabilized with eBioscience Foxp3/Transcription factor staining buffer (cat #00-5523-00), then stained with anti-pS6 (S235/236) Alexa 647 (clone D57.2.2E; Cell Signaling #5316S).

### Mice

Mice with a foxed Pik3ip1 gene (Pik3ip1^{fl/fl}) were generated by inGenious Targeting Laboratory Inc., using C57BL/6 ES cells. CD4-Cre mice on a C57BL/6 background were originally purchased from Taconic, then maintained by breeding to C57BL/6J mice (Jackson), and were used as controls. All mouse strains were on a C57BL/6 background and maintained in facilities of the University of Pittsburgh Division of Laboratory Animal Resources. Mice were age-matched within experiments, with approximately equal numbers of male and female animals. All mouse studies were performed in accordance with University of Pittsburgh Institutional Animal Care and Use Committee procedures.

### T Cell Purification and Differentiation

CD4 T cells from spleens and lymph nodes of naïve mice were purified by magnetic separation using the Miltenyi Biotec naïve CD4 T cell isolation kit (Cat#130-104-453). The purity of the final cell population was >90%. T cells were activated with plate-bound anti-CD3 (clone 145-2C11; Bio X Cell InVivoMab BE0001-1) in complete (RPMI medium supplemented with 10% bovine growth serum, 2 mM L-glutamine, 100 U/ml penicillin, 100 μg/ml streptomycin, 50 μM 2-mercaptoethanol, HEPES and sodium pyruvate). For Th1 differentiation, cells were cultured in the presence of recombinant mouse IL-12 (10 ng/ml), anti-IL-4 (10 μg/ml) and recombinant human IL-2 (50 U/ml). For Th17 differentiation, cells were cultured in the presence of recombinant human TGF-B (2.5 ng/ml), recombinant mouse IL-6 (20 ng/ml), and anti-IFNγ (10 μg/ml), anti-IL-4 (10 μg/ml), anti-IL-2 (20 μg/ml) neutralizing antibodies. For iTreg differentiation, cells were cultured in the presence of anti-IL-4 (10 μg/ml) and anti-IFNγ (10 μg/ml) neutralizing antibodies as well as recombinant human TGFB2 (10 ng/ml) and recombinant human IL-2 (50 U/ml). Cells were cultured for 3 days under Th differentiation conditions and then stimulated for 4 h with PMA (50 ng/ml) and ionomycin (1.34M) in the presence of golgi plug (BD biosciences cat#51-2301KZ). Cells were then harvested and analyzed by flow cytometry.

T helper cell differentiation reagents: Mouse IL-12 (Miltenyi Biotec Cat#130-096-707). Mouse IL-6 (Miltenyi Biotec Cat#130-094-065). TGF-β human recombinant (Sigma SRP3170-5UG). Anti-mouse IL-4 clone 11B11 (Bio X Cell #BE0045). Anti-mouse IL-2 clone S4B6-1 (Bio X Cell #BE0043). Anti-mouse IFN-γ clone XMG1.2 (Bio X Cell #BE0055).

The following antibodies and dyes were used for flow cytometry: Anti-mouse CD4 Brilliant Violet 510 clone GK1.5 (BioLegend #100449). Ghost Red 780 viability dye (TONBO biosciences #13-0865-T100). V450-rat anti-mouse Foxp3 Clone MF23 (BD Horizon #561293). mouse anti-mouse RORγt PE (clone Q31-378; BD Biosciences 562607). Anti-mouse IL-17A PerCP-Cy5.5 (clone eBio17B7; eBioscience 45-7177-80). Anti-mouse Foxp3 APC (clone FJK-16s; eBioscience 17-5773-82). Anti-mouse T-bet eFluor 660 (clone eBio4B10; eBioscience 50-5825-82). Anti-mouse IFN-γ PE-Cy7 (clone XMG1.2; Tonbo Biosciences 60-7311-U025). Anti-mouse CD25 FITC (clone PC61.5; Tonbo Biosciences 35-0251-U100).

### T Cell Activation

Splenocytes and lymphocytes obtained from conditional KO and WT mice were stimulated in complete RPMI with 3 μg/ml biotinylated anti-CD3 and anti-CD28 in the presence of 15 μg/ml streptavidin (anti-mouse CD3e biotin, clone 145-2C11, Tonbo Biosciences #30-0031-U500; hamster anti-mouse CD28 biotin, clone 37.51, BD Biosciences #553296; streptavidin, cat#189730, MilliporeSigma). Cells were activated for 15 mins, 30 mins, 60 mins or 4 hours. Activation was stopped by placing cells on ice, adding 1 ml of cold PBS and centrifugation, to settle cells, followed by aspiration of media.

The following antibodies and dyes were used for flow cytometry: Anti-mouse CD4 Brilliant Violet 510 (clone GK1.5; BioLegend #100449). Anti-mouse CD8a v450 (clone 53-6.7; Tonbo Biosciences 75-0081-U100). Anti-pS6 (S235/236) Alexa 647 (clone D57.2.2E; Cell Signaling #5316S). Hamster anti-mouse CD69 FITC (clone H1.2F3; BD Biosciences #557392). Ghost Red 780 viability dye (Tonbo Biosciences #13-0865-T100).

qPCR

RNA was extracted using the Qiagen RNeasy Mini Kit (cat#74106) and reverse-transcribed to generate cDNA with the Applied Biosystems High Capacity cDNA Reverse Transcription Kit (cat#4368813). Quantitative real-time polymerase chain reaction assays were performed with Power SYBR Green PCR Master Mix (Applied Biosystems cat#4367659) on a Step-One Plus Real Time PCR system. The abundance of TrIP mRNA was normalized to that of mGAPDH as calculated with the 2^{-ΔΔCT} method. The primers used are as follows:

Forward:
(SEQ ID NO: 15)
5′-ATGCTGTTGGCTTTGGGTACAC-3′;
Reverse:
(SEQ ID NO: 16)
5′-CGGCAGTAGTTGTGGTTGC-3′.

### Flow Cytometry

Before staining, cells were washed in staining buffer (1% bovine growth serum supplemented PBS). For extracellular staining, cells were stained at 4° C. with antibodies resuspended in staining buffer. For intracellular staining, cells were washed three times to remove excess extracellular staining and then fixed and permeabilized with either the eBioscience Foxp3/Transcription factor staining buffer set (cat #00-5523-00) or the BD Biosciences cytofix/perm kit (cat#554714) for transcription factor and cytosol analysis, respectively. Fixed and permeabilized cells were then stained at 4° C. with antibodies resuspended in 1× permeabilization buffer from the respective kits.

### Western Blotting Analysis and Immunoprecipitations

Cells were lysed in ice-cold NP-40 lysis buffer (1% NP-40, 1 mM EDTA, 20 mM tris-HCL (pH 7.4), 150 mM NaCl) for both protein analysis and immunoprecipitation. Immunoprecipitation was performed by mixing lysate with 20 μl of M2 anti-flag agarose beads for 3 hrs at 4 deg. C. Proteins were eluted from beads by mixing Lysate with 1×SDS (containing 5% B-mercaptoethanol) and boiling at 95 deg. C. for 10 mins. For western blot analysis, proteins were resolved by 10% SDS-polyacrylamide gel electrophoresis and were transferred onto polyvinylidene difluoride membranes which were then blocked in 4% BSA. The membranes were then incubated with the primary antibodies overnight. This was followed by incubating the membrane with HRP-conjugated secondary antibodies for 2 hours before detection with the SuperSignal West Pico ECL substrate (Thermo Fisher Scientific) and imaging on a Protein Simple FluorChem M.

Western Blot and CoIP Reagents:

Anti-HA (12CA5) (Roche Cat#11-583-816-001). Anti-Flag M2 Affinity Gel (Sigma Cat# A2220). Direct-Blot HRP anti-DYKDDDDK (Clone L5; BioLegend cat#637311).

### Listeria Infection

Control (CD4-Cre alone) and Pik3ip1^{fl/fl}×CD4-Cre mice were infected intravenously with 15,000 CFU LM-GP33 resuspended in 200 μl of PBS. Bacterial titers were quantified by lysing of whole livers in PBS and plating of a 10-fold dilution of bacteria on brain-heart infusion agar plates overnight. Immune response to LM-GP33 infection was quantified by extracting splenocytes and staining with a GP33 specific tetramer in complete RPMI at room temperature for 1 hour. After Tetramer staining, cells were stained with the following antibodies and dyes and analyzed by flow cytometry: Ghost Red 780 viability dye (Tonbo Biosciences #13-0865-T100), anti-mouse CD4 Brilliant Violet 510 (clone GK1.5; BioLegend #100449), anti-mouse CD8a PE (clone 53-6.7; Tonbo Bioscience 50-0081-U100), anti-mouse CD62L PerCP-Cy5.5 (clone MEL-14; eBioscience 45-0621-80), Anti-mouse KLRG1 FITC (clone 2F1; Tonbo Biosciences 35-5893-U100), anti-mouse CD127 PE-Cy7 (clone A7R34; Tonbo Biosciences 60-1271-U025), and anti-mouse CD44 violetFluor 450 (clone IM7; Tonbo Biosciences 75-0441-U025). All Ab staining was performed at 4° C. in PBS containing 1% BGS and 0.1% sodium azide. GP33 tetramers were provided as APC fluorophore-conjugated tetramers by the NIH tetramer core facility and used for identification of gp33-specific CD8⁺ and CD4⁺ T cells.

### Results

### TrIP Inhibits PI3K/mTOR Pathway Signaling

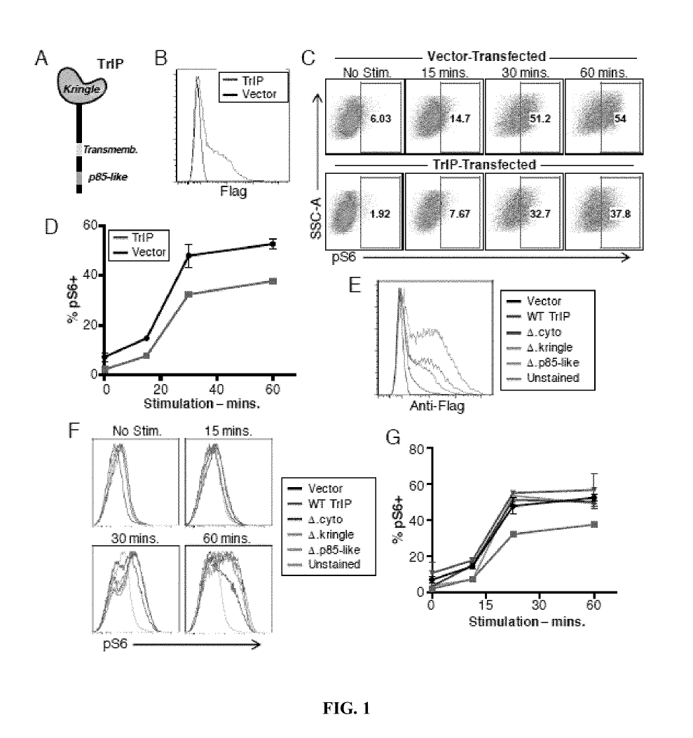

It was previously shown that ectopic expression of TrIP (also known as PIK3IP1) in T cells can inhibit the phosphorylation of Akt and thus its activation (DeFrances et al., 2012). A more sensitive readout of PI3K/Akt/mTOR activity is analysis of ribosomal protein S6 phosphorylation (pS6) by flow cytometry. In order to study the structural requirements of TrIP for modulation of T cell function, pS6 was evaluated in D10 T cells, a cell line with apparently normal PI3K signaling (Kane et al., 2004), in the context of ectopically expressed WT or mutant TrIP. The domain structure of TrIP is illustrated in FIG. 1A. D10 T cells were transfected with control plasmid or Flag-tagged TrIP (WT TrIP) and monitored TrIP expression using α-Flag antibody (FIG. 1B). Co-transfection of GFP was also used to determine transfection efficiency. One day after transfection, cells were stimulated with α-CD3/CD28 and analyzed at various time points (FIG. 1C-D). At all time points evaluated, cells with ectopic TrIP expression displayed lower pS6 compared with empty vector-transfected cells. It was also noted that, in the absence of stimulation, ectopic expression of TrIP resulted in lower basal pS6. These results support previous data suggesting that TrIP inhibits Akt activation in T cells (DeFrances et al., 2012; Menhart et al., 1995; Mikels et al., 2009; Patthy et al., 1984).

### The Kringle and Cytoplasmic Domains are Required for TrIP Activity

The importance of the kringle domain as a ligand-binding domain in other proteins (Menhart et al., 1995; Mikels et al., 2009; Patthy et al., 1984), and the degree of homology between the p85-like domain and the inter-SH2 domain of the p85 regulatory subunit of PI3K, suggests that these two domains may play important roles in the inhibition of PI3K by TrIP. To address this, Flag-tagged TrIP variants were designed lacking either the extracellular kringle domain (Δkringle-TrIP), the entire cytoplasmic region (Δcyto-TrIP) or only the p85-like domain (Δp85-like-TrIP). These constructs were transfected, along with a GFP transfection control, into D10 T cells (FIG. 1E) which were then stimulated with α-CD3/CD28 and analyzed by flow cytometry for S6 phosphorylation (FIG. 1F-G). While WT TrIP led to the attenuation of pS6, deletion of either the p85-like domain alone or the entire cytoplasmic region abolished the ability of TrIP to suppress pS6. Interestingly, expression of the kringle domain-deleted construct (which still possesses the p85-like domain) did not attenuate pS6. These results show that both the kringle and p85-like domains are essential for optimal TrIP inhibitory function.

### Cell-Surface TrIP is Downregulated Upon T Cell Activation

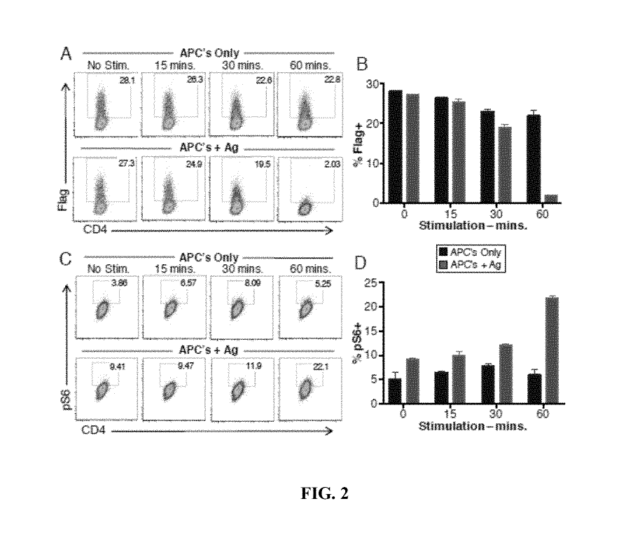

These data strongly support an inhibitory role for TrIP in T cells. Certain other negative regulators of T cell signaling (e.g. PTEN) are actively downregulated to promote T cell activation (Hawse et al., 2015; Newton and Turka, 2012), so it was examined how TCR activation might affect expression of TrIP protein. In order to replicate T cell stimulation in the presence of an antigen presenting cell (APC), the mouse B cell lymphoma line CH27 (Haughton et al., 1986) was used to stimulate D10 cells. CH27 cells were either pulsed with cognate antigen (chicken conalbumin) or left unpulsed, and then mixed with D10 T cells transfected with Flag-tagged PI3KIP1. There was a notable decrease over time in Flag-TrIP expression on the surface of D10 cells (FIG. 2A-B). Interestingly, this decreased expression corresponded with an increase of pS6 expression (FIG. 2C-D). As observed when D10 T cells were stimulated with α-CD3/CD28 (FIG. 1), antigen-pulsed CH27 cells induced a more immediate and robust pS6 signal in T cells transfected with empty vector, compared to those transfected with WT TrIP (not shown). These results support the model that T cells acutely modulate the expression of TrIP to promote the activation of TCR-induced PI3K signaling.

### Structure/Function Analysis of TrIP Cell Surface Expression and Inhibitory Activity

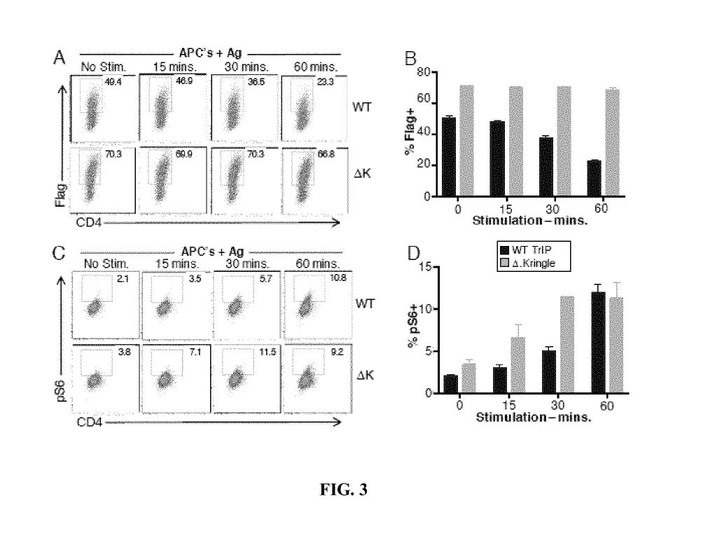

In order to probe the relevance of the kringle domain for TrIP expression and function, the CH27-D10 experiments were repeated with both Flag-tagged WT TrIP and Δkringle-TrIP-transfected D10 T cells and monitored Flag-TrIP expression and S6 phosphorylation. Since Δkringle-TrIP transfected cells showed a recovery in TCR-dependent pS6 (FIG. 1F-G), it was suspected that the absence of the kringle domain would lead to maintenance (and not downregulation) of TrIP. As shown above, D10 cells transfected with WT TrIP and stimulated with CH27 B cells alone (without antigen) did not show significant loss of TrIP expression over time. However, when mixed with antigen-pulsed CH27 cells, WT TrIP-expressing D10 T cells showed a significant loss of TrIP expression (FIG. 3A, top row and 3B). In contrast, D10 cells transfected with the Δkringle-TrIP mutant did not lose surface TrIP expression when stimulated with CH27 cells (with or without antigen) (FIG. 3A, bottom row and 3B). Consistent with the results above, upon mixing with antigen-pulsed CH27 cells, Δkringle-TrIP-expressing D10 T cells actually showed a more rapid increase in pS6 than cells transfected with WT TrIP (FIG. 3C-D). These results further confirm that the kringle domain is important for the inhibitory function of TrIP and shows the possibility that TrIP is regulated by interaction with a ligand.

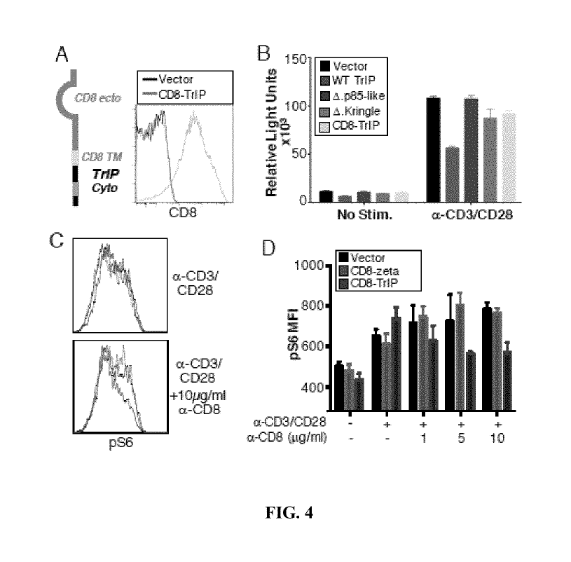

In the absence of a known ligand, a hCD8-TrIP chimera was designed to investigate possible effects of ligand engagement on TrIP function. The extracellular and transmembrane regions of human CD8 were fused with the cytoplasmic tail of TrIP, expressed in D10 T cells and detected at the cell surface with hCD8 antibody (FIG. 4A). Using a luciferase reporter driven by the NFAT promoter, it was observed that expression of hCD8-TrIP on D10 T cells was not sufficient to inhibit TCR signaling (FIG. 4B). However, upon crosslinking of hCD8-TrIP with varying concentrations of α-hCD8, a decrease in anti-CD3/CD28-induced pS6 was observed (FIG. 4C-D). This was in contrast to the effects of a previously described CD8-CD3 chimeric construct (Irving and Weiss, 1991), which upon crosslinking with anti-hCD8, modestly enhanced pS6 expression (FIG. 4C-D). These results show that binding of ligand to the kringle-domain may regulate TrIP function, possibly by aggregation of the protein and increased sequestration of PI3K from the TCR signaling machinery.

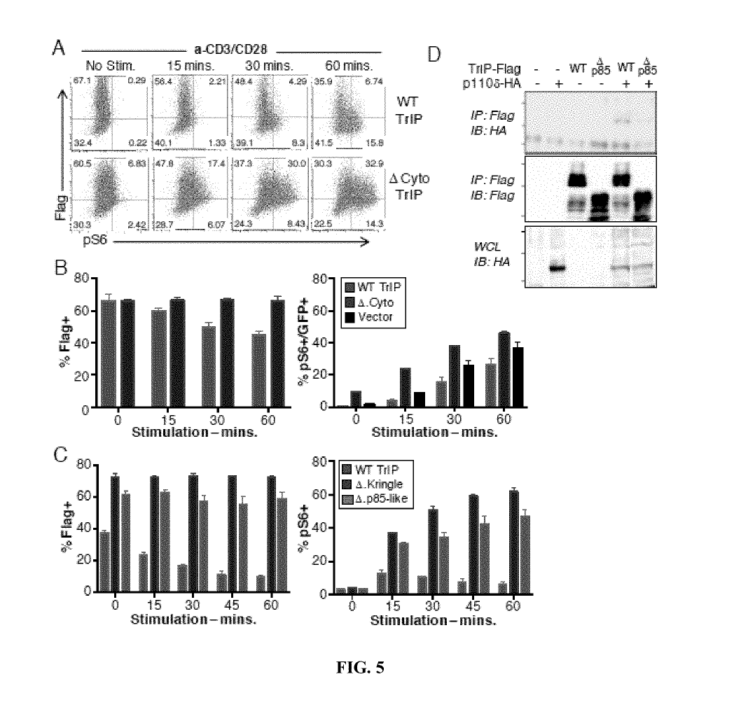

To determine whether inducible downregulation of TrIP only occurs in the context of stimulation by antigen and APCs (which may express a ligand for TrIP), transfected D10 cells were stimulated with α-CD3/CD28 and evaluated the surface expression of TrIP at various time points in the absence of APC and cognate antigen using the α-Flag antibody. Thus, it was observed that even after CD3/CD28 Ab stimulation, without APC's, WT TrIP expression was still downregulated (FIG. 5A-B) with a concomitant upregulation of pS6 (FIG. 5B). In contrast, neither Δcyto-TrIP (FIG. 5A-B), Δkringle-TrIP or Δp85-TrIP (FIG. 5C) expression was downregulated over the course of the stimulation by CD3 and CD28 Ab's. These data show that one or more ligands responsible for TrIP inhibition of PI3K may be located on the T cells themselves and interacts with PIK3P1 in a kringle domain-dependent fashion. However, this does not rule out the presence of an identical or different ligand on the APC's.

The p85-Like Domain of TrIP Interacts with p110δ PI3K

TrIP has been shown to interact with the PI3K catalytic subunits p110α/β in mouse embryonic fibroblast (MEF) cells, via a ‘p85-like’ domain with approximately 80% homology to the inter-SH2 domain of the PI3K regulatory subunit p85 (Zhu et al., 2007). However, it is not known whether TrIP can also interact with p110δ, which is the main catalytic subunit of PI3K activated by TCR signaling (Okkenhaug and Vanhaesebroeck, 2003). To test a possible interaction of p110δ with the p85-like domain of TrIP, 293 cells were transiently transfected with Flag-tagged WT TrIP or Δp85-TrIP, along with HA-tagged p110δ and performed co-IP and western blot analysis (FIG. 5D, top panel). Thus, WT TrIP could co-IP p110δ-PI3K; however, there was a significant reduction in the ability of Δp85-TrIP to co-IP p110δ (FIG. 5D top panel, last lane). These results are consistent with a previous report that examined the interaction of TrIP with other p110 isoforms in fibroblasts (Zhu et al., 2007), and show that TrIP may inhibit T cell activation through effects on p110δ.

### Deletion of TrIP Leads to Dysregulation of Primary T Cell Activation

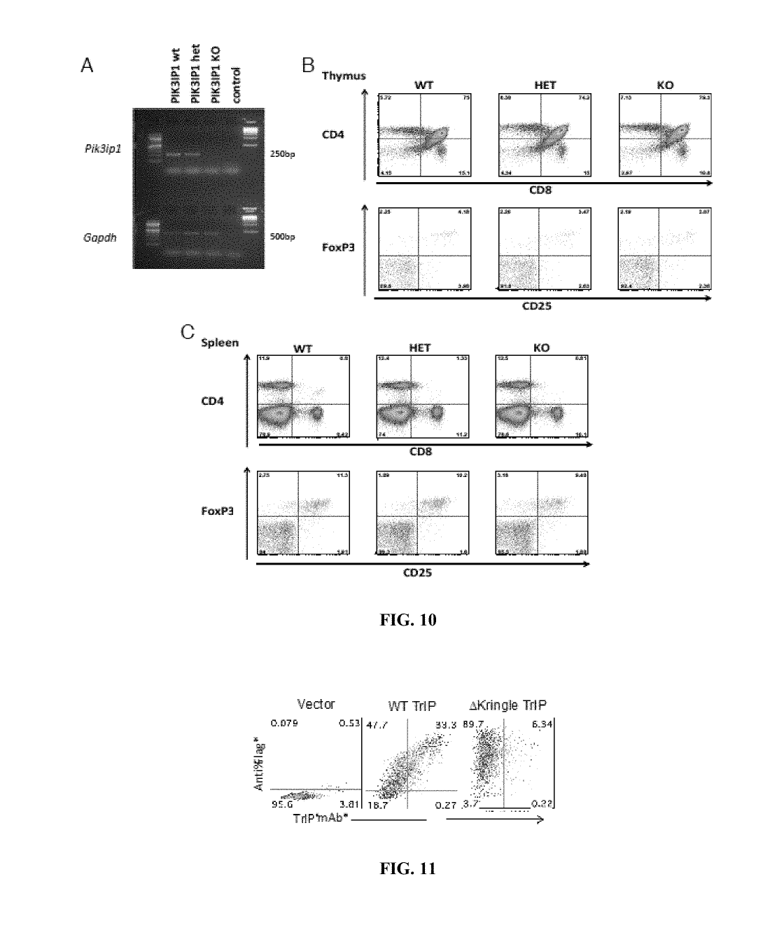

In order to assess the function of TrIP in T cells in vivo, mice were generated with LoxP-flanked (floxed) Pik3ip1 alleles (Pik3ip1^{fl/fl}) and bred them to mice with a CD4-driven Cre recombinase transgene. Mice with CD4-Cre alone were used as controls. T cells from spleen and lymph nodes of naïve homozygous (CD4-Cre×Pik3ip1^{fl/fl}) and heterozygous (CD4-Cre×Pik3ip1^{fl/wt}) TrIP conditional knockout mice were screened by RT-PCR for TrIP mRNA expression and compared to wild type mice (CD4-Cre Pik3ip1^{wt/wt}) (FIG. 10). Analysis of thymus, spleen and lymph nodes from these mice showed similar percentages of CD4 and CD8 T cells in all compartments as well as normal numbers of nTregs (Foxp3⁺CD25⁺) showing that T cell development was normal (FIG. 10). Absolute cell numbers from these compartments were also similar across all strains (data not shown) as was the gross size of primary (thymus) and secondary (spleen and lymph nodes) lymphoid organs (data not shown). These data show that T cell-specific deletion of TrIP does not grossly affect T cell development or homeostasis.

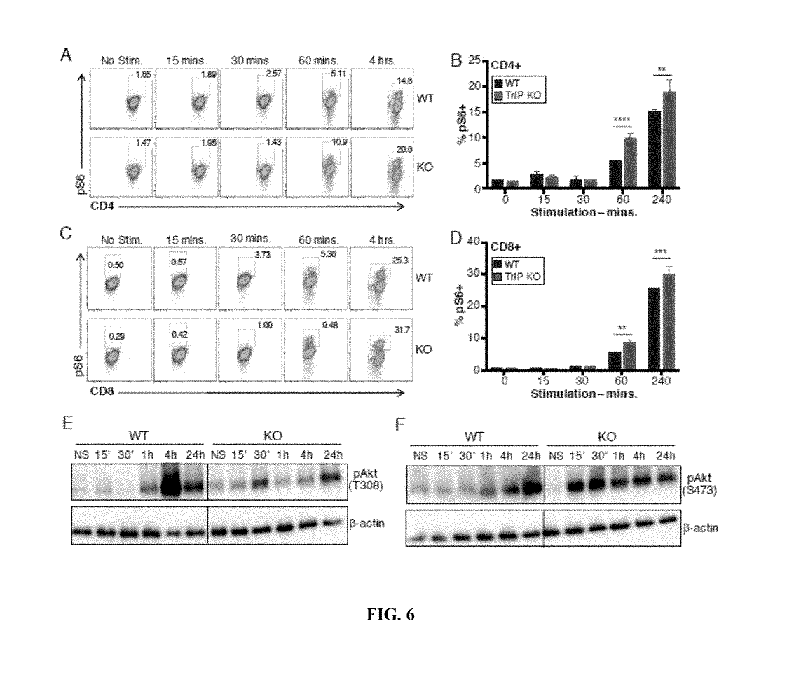

Based on data shown above, the effects of deleting TrIP in primary T cells was analyzed to determine if this would lead to enhanced TCR signaling. Lymphocytes were stimulated from wild type (WT) and conditionally-deleted TrIP (KO) age-matched mice with α-CD3/CD28 for varying times and evaluated early T cell activation by pS6 and CD69 expression. Following stimulation, TrIP KO CD4⁺ and CD8⁺ T cells showed significantly higher pS6 at later time points (1-4 hrs.) (FIG. 6A-D). CD69, an early activation marker that can typically be observed approximately four hours after stimulation, was expressed more highly in KO T cells compared to wild type cells (not shown). These data are consistent with the observation of more robust phosphorylation of Akt at both T308 and S473 in peripheral T cells lacking TrIP (FIG. 6E-F). Taken together, these results demonstrate that the loss of TrIP in T cells promotes more efficient TCR signaling and early T cell activation, consistent with the effects of an inhibitory protein.

Deletion of TrIP Enhances Th1 T Cell Polarization and Inhibits iTreg Generation

Previous research suggested that higher PI3K signaling may lead to enhanced Th1 potential, while lower levels of PI3K would favor generation of iTregs (He et al., 2008; Sauer et al., 2008). Based on these findings, T cell polarization towards a pro-inflammatory phenotype was investigated in TrIP KO T cells. Naïve CD4⁺ T cells were isolated from WT and KO mice and cultured them with α-CD3/CD28 mAbs for three days under neutral (no polarization), Th0 (suppression of all polarization), Th1, Th17 and iTreg skewing conditions. At the end of culture, cells were re-stimulated with PMA/ionomycin for four hours and then evaluated for Th1 (T-bet and IFN-γ), Th17 (RORγt and IL17a) and iTreg (CD25 and Foxp3) phenotype. Thus, a significant increase in the number of cells producing IFN-γ in TrIP KO T cell cultures was noted, compared to WT cells, especially under Th0 and Th1, and even iTreg, conditions (FIG. 7A). Interestingly, a smaller, but reproducible, decrease was observed in the number of TrIP KO T cells making IL-17a, specifically under Th17 polarization conditions (FIG. 7B). More strikingly, TrIP knockout T cells cultured under iTreg-generating conditions were significantly less efficient at producing Foxp3⁺CD25⁺ iTreg (FIG. 7C-D). This result is consistent with previous findings that PI3K/Akt/mTOR signaling suppresses the generation of iTreg (Sauer et al., 2008). Interestingly, when the expression of Pik3ip1 mRNA was assessed after culturing under the various Th polarization conditions, it was found that Pik3ip1 was severely downregulated in Th0 and Th1 cells. By contrast, Pik3ip1 was maintained at a somewhat higher level in iTreg, although still not as high as naïve cells (FIG. 7E), which is consistent with an apparent requirement to suppress PI3K signaling in Treg.

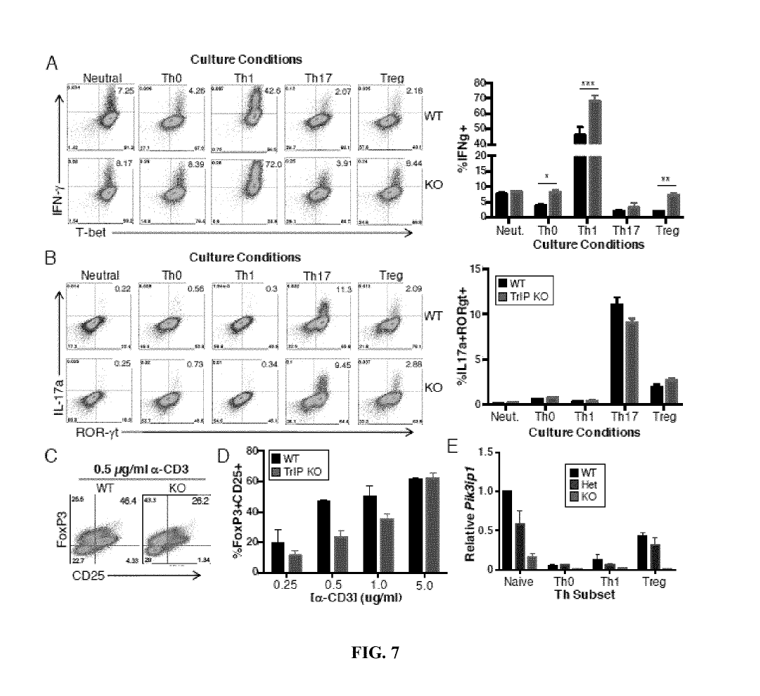

In order to determine whether the difference in Th1 polarization was actually due to the absence of PI3K modulation by TrIP, the Th1 differentiation assay was performed on WT and KO T cells with the addition of moderate concentrations of PI3K/Akt pathway inhibitors. Specifically, an Akt1/2 inhibitor (Akti-1/2), a pan-PI3K inhibitor (LY294002) and a selective p110δ inhibitor (IC-87114) were used. Cells were stimulated with α-CD3/CD28 as in FIG. 7 and cultured under Th1 conditions in the presence of varying concentrations of the PI3K inhibitors. At the end of culture, cells were re-stimulated with PMA/Ionomycin and evaluated by flow cytometry for IFN-γ expression. It was found that IFN-γ production by KO cells was significantly inhibited at concentrations as low as 0.5 μM with all three of the inhibitors tested (FIG. 8A-B). Consistent with p110δ being the most prevalent catalytic subunit of PI3K linked to TCR signaling, IC-87114 was the most potent inhibitor at all concentrations. Overall, a reduction of IFN-γ production was observed by both WT and TrIP KO T cells in the presence of inhibitors, but this inhibition was more distinct with the KO cells. These results support the idea that the high IFN-γ production by TrIP KO T cells is due to a loss of TrIP inhibition of PI3K.

### Enhanced In Vivo Function of T Cells Lacking TrIP

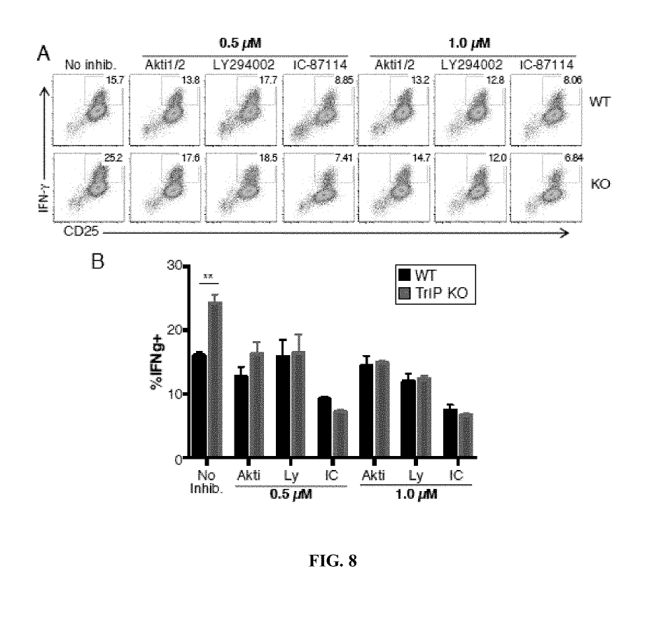

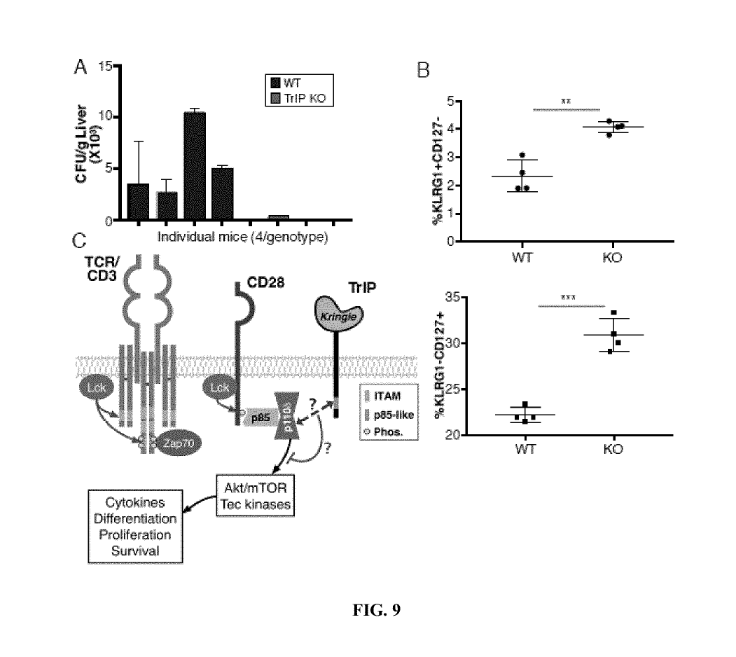

To explore the role of TrIP function in immune challenged mice, mice were infected with Listeria monocytogenes, a disease model that requires effective CD8⁺ and Th1 T cell responses for clearance and memory generation (Lara-Tejero and Pamer, 2004; Pamer, 2004). Specifically, a recombinant Listeria monocytogenes strain (LM-GP33) was used that expresses the GP(33-41) epitope from LCMV (Kaech and Ahmed, 2001). Mice were infected with a relatively high titer (15,000 CFU) of LM-GP33, as TrIP KO mice would be able to clear the infection more efficiently, based on the in vitro data detailed above. Thus, while all WT mice had culturable bacteria in the liver by day four after infection, all but one KO mouse had apparently cleared the infection by this point, at least below the limit of detection of the assay (FIG. 9A). The one KO mouse that did have detectable bacterial burden had significantly less than all other WT mice. The T cell response of these mice during the infection was evaluated next, and it was found that there was no significant difference in the percentage of LM-GP33-specific CD8⁺ T cells, based on tetramer staining (not shown). However, a significantly higher percentage of short-lived effector cells (CD127^{lo}KLRG1^{hi}) and memory precursor effector cells (KLRG1^{lo}CD127^{hi}) within the CD8⁺ T cell compartment were observed (FIG. 9B). Thus, the enhanced sensitivity of TrIP KO T cells described above does indeed translate to more robust in vivo activity of these cells in response to an intracellular bacterial infection.

### DISCUSSION

TrIP is a transmembrane protein that contains two identifiable domains: an extracellular kringle domain, implicated in ligand interaction in related proteins (Ji et al., 1998; Menhart et al., 1995; Patthy et al., 1984), and an intracellular ‘p85-like’ domain. An initial study showed that PI3K was inhibited by TrIP through its interaction with the catalytic subunit of PI3K via the p85-like domain (Zhu et al., 2007). It was also shown that silencing TrIP in T cells leads to an upregulation in T cell signaling (DeFrances et al., 2012). However, the role of the kringle domain in TrIP function remains largely unknown. With recent studies implicating a loss of function or expression of TrIP in some cancers (He et al., 2008; Wong et al., 2014), a more complete understanding of TrIP structure and function in an immune context was explored. In order to ascertain the molecular mechanism through which TrIP is regulated, Flag-tagged variants of TrIP were expressed in T cells. These results showed that overexpression of WT TrIP in D10 cells resulted in a slower rate of phosphorylation of ribosomal S6 protein, further validating the observation that TrIP inhibits the PI3K/Akt pathway. Also, this example provides the first direct evidence that the p85-like domain is important for the inhibitory function of TrIP. Thus, in contrast to WT TrIP, ectopic expression of TrIP mutants lacking either the entire cytoplasmic region or just the p85-like domain did not inhibit T cell signaling.

In this example, the inhibitory activity of TrIP was inversely correlated with surface expression of the protein, as only when TrIP expression was mostly downregulated, was a significant upregulation of pS6 observed, showing TrIP expression must be downregulated in order for T cell signaling to proceed following TCR activation. While the mechanisms behind this downregulation are still not clear, there is precedent for acute down-regulation of other immune checkpoint molecules. Thus, Lag3 and Tim-3 can be inducibly cleaved from the cell surface by metalloproteases, resulting in enhanced T cell activation (Clayton et al., 2015; Li et al., 2007), while PTEN, an intracellular inhibitor of the PI3K pathway, is downregulated by multiple mechanisms, including post-translational modification and degradation (Hawse et al., 2015). In addition, after long periods of stimulation, Pik3ip1 message is also downregulated in effector T cells and maintained at higher levels in iTreg.

It has been reported that kringle domains in other proteins can serve regulatory functions, including acting as sites for protein-protein interactions (Cao et al., 2002; Castellino and McCance, 1997; Thery and Stern, 1996; Tolbert et al., 2010). Thus, the kringle domain could serve to modulate the activity of TrIP. Indeed, as with the p85-like domain, the kringle domain appears to be essential for TrIP inhibitory function. Overexpression of a TrIP construct in which the kringle domain was deleted led to higher induction of pS6 (after either Ab or Ag/APC stimulation) than was observed in T cells transfected with WT TrIP or even cells transfected with empty vector, suggesting that this construct may function as a “dominant negative,” to inhibit endogenous TrIP. Furthermore, substitution of the kringle domain with the extracellular region of hCD8, and subsequent crosslinking by α-CD8, led to diminished induction of pS6 after TCR stimulation. It is shown here that cell-surface TrIP expression is downregulated during the course of TCR stimulation, even in the absence of APCs, and only occurs with WT TrIP and not truncated variants, leading to the conclusion that one or more ligands that activates TrIP inhibitory activity is present on T cells. How binding of a ligand, expressed by either an APC or the T cell itself, might regulate TrIP function is not clear at this point. One possibility is that the kringle domain is required for recruitment of TrIP into proximity of the TCR and/or CD28 during synapse formation. Another non-exclusive possibility is that ligand binding may cause a conformational change in TrIP which promotes the binding, and inactivation, of PI3K.

While no obvious developmental defects in T cell compartments in mice with T cell-specific TrIP deficiency were observed, the possibility that TrIP may play a more subtle role in TCR repertoire selection cannot be ruled out. When stimulated, TrIP knockout T cells exhibited more robust, early activation and displayed higher Th1 and lower iTreg differentiation potential. The altered T cell lineage skewing in TrIP KO mice appeared to be a result (at least in part) of increased PI3K signaling, as PI3K inhibitors were able to reverse this phenotype. In addition, the increased tendency of TrIP KO T cells to make IFN-γ could impact the immune response to an infection. Analysis of LM-GP33 infected mice seven days after infection showed no significant differences between wild type and knockout mice (data not shown). This could be due to the fact that C57Bl/6 mice are generally able to clear Listeria monocytogenes and mount a peak T cell response by day seven. Analysis at day four, however, showed that TrIP knockout mice had significantly higher percentages of both SLECs and MPECs, suggesting that in the absence of TrIP, these mice mounted a more robust T cell response. Previous work by others has shown that Listeria bacteria replicate for three days after infection of mice (Wong and Pamer, 2003). Here, it was observed that by day four, TrIP knockout mice had cleared the infection, compared to wild type mice that still had significant bacterial burden. Based on the typical immune response to L. monocytogenes infection (Pamer, 2004; Wong and Pamer, 2003), clearance of bacteria correlates with the peak of the immune response. It is possible that with the increased T cell signaling in the absence of TrIP, CD4⁺ T cells are able to recruit more APCs for priming of CD8⁺ T cells which results in faster clearance.

Overall, the results point to TrIP as a target for modulating immune responses under conditions of chronic infection, cancer or autoimmunity. In the former settings, briefly turning off or reducing the TrIP inhibitory pathway allows for a more effective clearance of pathogens or tumors. Conversely, triggering an upregulation of TrIP activity or expression can combat pathological immune responses such as autoimmune diseases.

### REFERENCES CITED IN THIS EXAMPLE

Agoulnik, I. U., M. C. Hodgson, W. A. Bowden, and M. M. Ittmann. 2011. INPP4B: the new kid on the PI3K block. Oncotarget 2:321-328.
Antignano, F., M. Ibaraki, C. Kim, J. Ruschmann, A. Zhang, C. D. Helgason, and G. Krystal. 2010. SHIP is required for dendritic cell maturation. Journal of immunology (Baltimore, Md.: 1950) 184:2805-2813.
Cantley, L. C. 2002. The phosphoinositide 3-kinase pathway. Science (New York, N.Y.) 296:1655-1657.
Cao, Y., R. Cao, and N. Veitonmaki. 2002. Kringle structures and antiangiogenesis. Curr Med Chem Anticancer Agents 2:667-681.
Carracedo, A., and P. P. Pandolfi. 2008. The PTEN-PI3K pathway: of feedbacks and cross-talks. Oncogene 27:5527-5541.
Castellino, F. J., and S. G. McCance. 1997. The kringle domains of human plasminogen. Ciba Found Symp 212:46-60; discussion 60-45.
Christen, M. T., P. Frank, J. Schaller, and M. Llinas. 2010. Human plasminogen kringle 3: solution structure, functional insights, phylogenetic landscape. Biochemistry 49:7131-7150.
Clayton, K. L., M. B. Douglas-Vail, A. K. Nur-ur Rahman, K. E. Medcalf, I. Y. Xie, G. M. Chew, R. Tandon, M. C. Lanteri, P. J. Norris, S. G. Deeks, L. C. Ndhlovu, and M. A. Ostrowski. 2015. Soluble T cell immunoglobulin mucin domain 3 is shed from CD8+ T cells by the sheddase ADAM10, is increased in plasma during untreated HIV infection, and correlates with HIV disease progression. Journal of virology 89:3723-3736.
DeFrances, M. C., D. R. Debelius, J. Cheng, and L. P. Kane. 2012. Inhibition of T-cell activation by PIK3IP1. European journal of immunology 42:2754-2759.
Dillon, L. M., and T. W. Miller. 2014. Therapeutic targeting of cancers with loss of PTEN function. Current drug targets 15:65-79.
Engelman, J. A. 2009. Targeting PI3K signalling in cancer: opportunities, challenges and limitations. Nature reviews. Cancer 9:550-562.
Fresno Vara, J. A., E. Casado, J. de Castro, P. Cejas, C. Belda-Iniesta, and M. Gonzalez-Baron. 2004. PI3K/Akt signalling pathway and cancer. Cancer Treat Rev 30:193-204.
Fruman, D. A. 2007. The role of class I phosphoinositide 3-kinase in T-cell function and autoimmunity. Biochemical Society transactions 35:177-180.
Fruman, D. A., R. E. Meyers, and L. C. Cantley. 1998. Phosphoinositide kinases. Annu Rev Biochem 67:481-507.
Han, J. M., S. J. Patterson, and M. K. Levings. 2012. The Role of the PI3K Signaling Pathway in CD4(+) T Cell Differentiation and Function. Frontiers in immunology 3:245.
Haughton, G., L. W. Arnold, G. A. Bishop, and T. J. Mercolino. 1986. The CH series of murine B cell lymphomas: neoplastic analogues of Ly-1+ normal B cells. Immunological reviews 93:35-51.
Hawse, W. F., R. P. Sheehan, N. Miskov-Zivanov, A. V. Menk, L. P. Kane, J. R. Faeder, and P. A. Morel. 2015. Cutting Edge: Differential Regulation of PTEN by TCR, Akt, and FoxO1 Controls CD4+ T Cell Fate Decisions. Journal of immunology 194:4615-4619.
He, X., Z. Zhu, C. Johnson, J. Stoops, A. E. Eaker, W. Bowen, and M. C. DeFrances. 2008. PIK3IP1, a negative regulator of PI3K, suppresses the development of hepatocellular carcinoma. Cancer research 68:5591-5598.
Irving, B. A., and A. Weiss. 1991. The cytoplasmic domain of the T cell receptor zeta chain is sufficient to couple to receptor-associated signal transduction pathways. Cell 64:891-901.
Ji, W. R., F. J. Castellino, Y. Chang, M. E. Deford, H. Gray, X. Villarreal, M. E. Kondri, D. N. Marti, M. Llinas, J. Schaller, R. A. Kramer, and P. A. Trail. 1998. Characterization of kringle domains of angiostatin as antagonists of endothelial cell migration, an important process in angiogenesis. FASEB journal: official publication of the Federation of American Societies for Experimental Biology 12:1731-1738.
Kaech, S. M., and R. Ahmed. 2001. Memory CD8+ T cell differentiation: initial antigen encounter triggers a developmental program in naïve cells. Nature immunology 2:415-422.
Kane, L. P., M. N. Mollenauer, and A. Weiss. 2004. A proline-rich motif in the C terminus of Akt contributes to its localization in the immunological synapse. Journal of immunology 172:5441-5449.
Lara-Tejero, M., and E. G. Pamer. 2004. T cell responses to Listeria monocytogenes. Curr Opin Microbiol 7:45-50.
Li, N., Y. Wang, K. Forbes, K. M. Vignali, B. S. Heale, P. Saftig, D. Hartmann, R. A. Black, J. J. Rossi, C. P. Blobel, P. J. Dempsey, C. J. Workman, and D. A. Vignali. 2007. Metalloproteases regulate T-cell proliferation and effector function via LAG-3. The EMBO journal 26:494-504.
Menhart, N., G. J. Hoover, S. G. McCance, and F. J. Castellino. 1995. Roles of individual kringle domains in the functioning of positive and negative effectors of human plasminogen activation. Biochemistry 34:1482-1488.
Mikels, A., Y. Minami, and R. Nusse. 2009. Ror2 receptor requires tyrosine kinase activity to mediate Wnt5A signaling. The Journal of biological chemistry 284:30167-30176.
Newton, R. H., and L. A. Turka. 2012. Regulation of T cell homeostasis and responses by pten. Frontiers in immunology 3:151.
Okkenhaug, K., and B. Vanhaesebroeck. 2003. PI3K-signalling in B- and T-cells: insights from gene-targeted mice. Biochemical Society transactions 31:270-274.
Pamer, E. G. 2004. Immune responses to Listeria monocytogenes. Nature reviews. Immunology 4:812-823.
Patthy, L., M. Trexler, Z. Vali, L. Banyai, and A. Varadi. 1984. Kringles: modules specialized for protein binding. Homology of the gelatin-binding region of fibronectin with the kringle structures of proteases. FEBS letters 171:131-136.
Sauer, S., L. Bruno, A. Hertweck, D. Finlay, M. Leleu, M. Spivakov, Z. A. Knight, B. S. Cobb, D. Cantrell, E. O'Connor, K. M. Shokat, A. G. Fisher, and M. Merkenschlager. 2008. T cell receptor signaling controls Foxp3 expression via PI3K, Akt, and mTOR. Proceedings of the National Academy of Sciences of the United States of America 105:7797-7802.
Thery, C., and C. D. Stern. 1996. Roles of kringle domain-containing serine proteases in epithelial-mesenchymal transitions during embryonic development. Acta Anat (Basel) 156:162-172.
Tolbert, W. D., J. Daugherty-Holtrop, E. Gherardi, G. Vande Woude, and H. E. Xu. 2010. Structural basis for agonism and antagonism of hepatocyte growth factor. Proceedings of the National Academy of Sciences of the United States of America 107:13264-13269.
Wong, C. C., I. Martincorena, A. G. Rust, M. Rashid, C. Alifrangis, L. B. Alexandrov, J. C. Tiffen, C. Kober, C. Chronic Myeloid Disorders Working Group of the International Cancer Genome, A. R. Green, C. E. Massie, J. Nangalia, S. Lempidaki, H. Dohner, K. Dohner, S. J. Bray, U. McDermott, E. Papaemmanuil, P. J. Campbell, and D. J. Adams. 2014. Inactivating CUX1 mutations promote tumorigenesis. Nature genetics 46:33-38.
Wong, P., and E. G. Pamer. 2003. Feedback regulation of pathogen-specific T cell priming. Immunity 18:499-511.
Zhu, Z., X. He, C. Johnson, J. Stoops, A. E. Eaker, D. S. Stoffer, A. Bell, R. Zarnegar, and M. C. DeFrances. 2007. PI3K is negatively regulated by PIK3IP1, a novel p110 interacting protein. Biochemical and biophysical research communications 358:66-72.

### Example 2. Manipulation of T Cell and Mast Cell Activation and Differentiation by PIK3IP1

### Background

PI-3 kinases (PI3K) are a family of lipid kinases that are activated by numerous cellular receptors. Proper regulation of PI3K and its products is critical to cellular homeostasis (2-4). Activating mutations or amplification of PI3K and the downstream effector Akt have been implicated in various cancers (5-7). Conversely, at least two negative regulators of signaling downstream of PI3K function as tumor suppressors: the lipid phosphatases PTEN (5), and INPP4B (8). Downstream effectors of PI3K include many PH domain-containing proteins. Of particular relevance for antigen-responsive leukocytes like T cells and mast cells are the Tec family tyrosine kinases, which enhance PLC-γ1 activation, as well as the Akt family of serine/threonine kinases (4, 9), which regulate cell survival, metabolism, growth and protein translation (10-12).

After encountering their cognate antigen, naïve T cells become activated, a process that results in dramatic increases in cytokine production and proliferation. Depending on the cytokine milieu, they can differentiate down one of a number of specific paths, particularly in the case of CD4⁺“helper” T cells (Th). Th1 T cells enhance responses to intracellular pathogens like viruses and some bacteria; these cells secrete high levels of IFN-γ, and express the transcription factor Tbet. Th2 T cells help combat parasitic infections and contribute to atopic diseases; these cells are characterized by expression of IL-4 and the transcription factor Gata-3. Regulatory T cells (Treg) limit T cell responses and prevent autoimmunity, and these cells express suppressive cytokines like IL-10 and the transcription factor FoxP3. PI3K signaling is a critical node downstream of TCR signaling, for control of Treg differentiation and function (13-15).

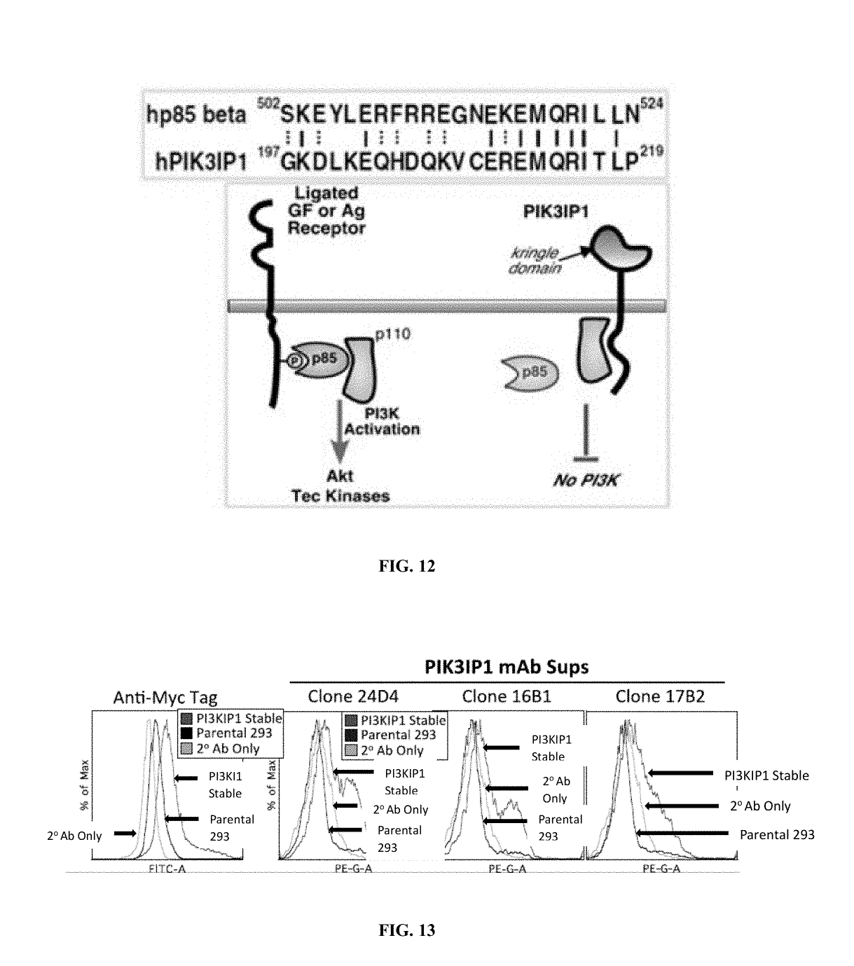

Recently another negative regulator of PI3K that acts more proximally to inhibit PI3K activity was described (1). PIK3IP1 is a transmembrane protein that contains an extracellular kringle domain and a cytoplasmic domain with a region homologous to the inter-SH2 p110-binding domain of p85 (FIG. 12, top). Although the precise mechanism is not clear, PIK3IP1 may sequester PI3K complexes away from growth factor receptors or, alternatively, may interfere with the allosteric mechanism of p110 activation that depends on associated p85 SH2-pTyr interactions (1) (FIG. 12, bottom). PIK3IP1 can also function as a tumor suppressor, since transgenic expression of PIK3IP1 in the liver suppressed the development of hepatocellular carcinoma (HCC) in a susceptible mouse strain (16). Importantly, PIK3IP1 is also expressed in humans, where it bears 80% identity to the mouse protein. Furthermore, deletion of the PIK3IP1 gene has been implicated in the development of certain types of testicular cancer (17). Based on analysis of gene expression databases, it was revealed that T cells and mast cells are the cell types that express the highest levels of Pik3ip1 mRNA. Importantly, gene expression data from human tissues is consistent with PIK3IP1 mRNA being highly expressed in both T cells and mast cells (not shown).

Intriguingly, it was found that PIK3IP1 (which they termed calcineurin-regulated kringle domain protein or CRKD) is down-regulated in mouse embryos by calcium signaling through the NFAT phosphatase calcineurin. Thus, Pik3ip1/Crkd mRNA is highly upregulated in mouse embryos deficient in calcineurin, while in humans PIK3IP1 protein is over-expressed in breast cancer (18). This group also reported a possible PIK3IP1/CRKD ligand, a transmembrane protein with an IgV domain, now known as Trem16 (18).

In addition to its role in tumorigenesis, the PI3K pathway plays a complex role in leukocyte function (2, 19). Therefore, it is important to understand how this pathway is regulated. PIK3IP1 functions in the regulation of T cell activation and development. In brief, it was discovered that PIK3IP1 message and protein are expressed in T cells. Furthermore, while ectopic expression of PIK3IP1 inhibits signaling pathways associated with T cell activation, decreasing expression of PIK3IP1 (with siRNA) augments the same pathways, at least in T cell lines.

### T Cells

As shown in FIG. 13, monoclonal antibodies to the ecto domain of mouse PIK3IP1 were developed, which are used in flow cytometry to identify T cells (if any) retaining surface expression of PIK3IP1 in inducible knockout animals. Such antibodies are also useful for mechanistic studies, where the effects of PIK3IP1 interaction with a ligand are investigated. These monoclonal antibodies (mAbs) are examined for blocking interaction of PIK3IP1 with this ligand, or whether any of the antibodies themselves possess agonistic activity.

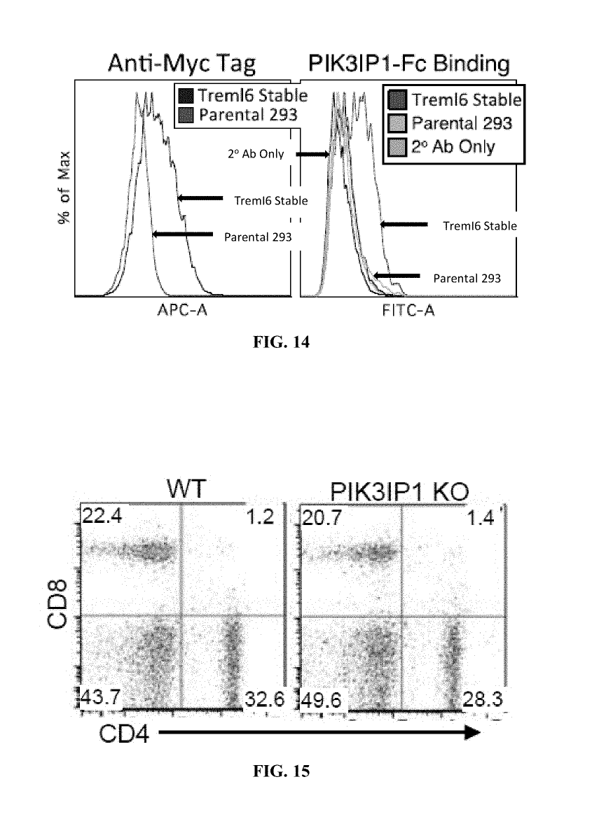

As described above, previous work identified a possible ligand for PIK3IP1, a transmembrane, Ig domain-containing protein now known as Trem16 (18). From the perspective of studying the regulation of T cell activation by PIK3P1, it is intriguing that this potential ligand is highly expressed in certain myeloid lineage cells, at least at the message level (BioGPS, ImmGen). A cDNA clone encoding Trem16 was obtained, which was cloned into a vector with a C-terminal Myc epitope tag. A stable clone of HEK293 cells expressing this Myc-tagged Trem16 was established (FIG. 14, left). These cells, or parental 293 cells, were stained with a PIK3IP1-Fc fusion protein. As shown in FIG. 14 (right), PIK3IP1-Fc specifically stained the Trem16 stable clone, but not parental 293 cells. An Fc fusion protein containing the ecto domain of Trem16 has also been generated (not shown).

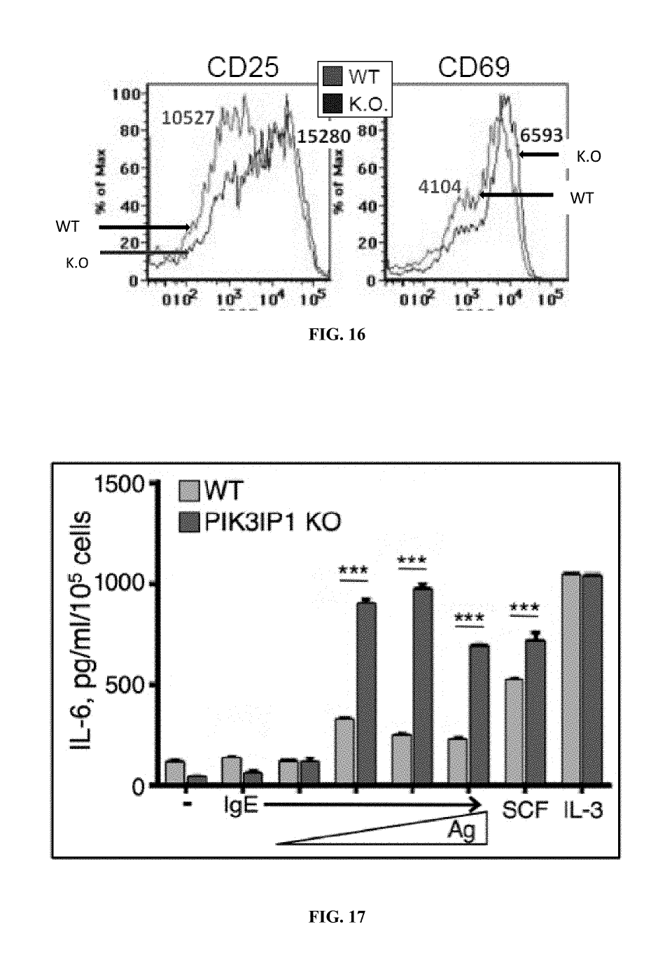

The effects on T cells of a knockout mouse model of PIK3IP1 deficiency was also analyzed. Initial analysis of PIK3IP1 inducible KO mice was conducted with a ubiquitously expressed Cre, driven by Sox2. Thymus, spleens and lymph nodes were harvested from these mice, to analyze T cell development. There were no overt effects of PIK3IP1 KO on numbers or proportions of T cell subsets in the thymus (not shown) or the periphery (FIG. 15). This finding is reminiscent of other KO mouse models of signaling proteins, like those of Itk, which initially presented with rather subtle phenotypes (21, 22), but later was revealed to have significant effects on specific aspects of T cell function (23-26). Splenic T cells from these mice were also isolated and stimulated in vitro with anti-CD3/CD28 Ab's and flow cytometry was performed to determine the relative upregulation of early markers of T cell activation (FIG. 16). An increase in relative upregulation of both CD25 (IL-2r alpha chain) and CD69 in PIK3IP1 KO T cells was observed (blue lines), compared with WT littermate controls (red lines). These findings suggest that PIK3IP1-deficient T cells have an enhanced sensitivity to TCR stimulation.

### Mast Cells

It was recently found that PIK3IP1 may negatively regulate the activation of mast cells, which use similar but distinct signaling pathways to control their responses to antigen. Mast cells in various tissues play key roles in type II immunity against parasites (aided by Th2 T cells), but can contribute to atopic diseases like allergic asthma, atopic dermatitis, etc. Like T cells, mast cells are activated by antigen, although mast cells encounter antigen indirectly, through pre-bound IgE, which then signals through FcεRI. Similar to T cells, mast cell activation requires antigen-dependent activation of PI3K (28).

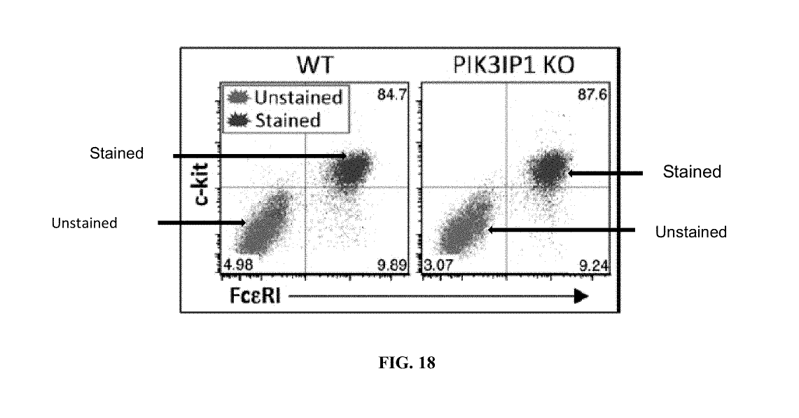

Mouse mast cells express high levels of Pik3ip1 mRNA, and may down-regulate this message and protein after mast cell activation. The initial analysis of inducible PIK3IP1 knockout mice used a ubiquitously expressed Sox2-Cre. Bone marrow was harvested from these mice and generated BMMC's by culturing with IL-3. Mature mast cells were obtained from bone marrow of PIK3IP1 KO mice, and these cells matured normally, as determined by expression of c-kit and FcεRI (FIG. 18). Mast cells were sensitized with DNP-specific IgE, then stimulated cells with increasing amounts of DNP-HSA. Cells were also stimulated with the growth factor SCF (stem cell factor) or with the cytokine IL-3, which had been used to drive differentiation of these cells. PIK3IP1 KO mast cells produced significantly more cytokine (IL-6) across a range of Ag concentrations (FIG. 17). A smaller, but statistically significant, increase in IL-6 was observed after stimulation with SCF, which signals through c-kit, while there was no difference after IL-3 stimulation. Thus, mast cells lacking PIK3IP1 are hyper-responsive to stimulation with IgE/Ag or SCF, consistent with a negative regulatory role for PIK3IP1.

### Summary

PIK3IP1 is a transmembrane protein and can be targeted (for inhibition or activation) with specific monoclonal antibodies, small molecules, or polypeptides. The presence of a small number (5-10) of other kringle domains in the genome also suggests that off-target effects can be less problematic than for reagents targeting more common structural domains. Thus, crosslinking of PIK3IP1 (by either a mAb or a small molecule) can inhibit T cell or mast cell activation in autoimmunity and/or atopy. In addition, antagonism of PIK3IP1 can enhance T cell immunity to tumors or for clearance of viral reservoirs.

### REFERENCES CITED IN THIS EXAMPLE

The disclosures of all articles and references, including patent applications and publications, are incorporated by reference for all purposes.
1. Zhu, Z., X. He, C. Johnson, J. Stoops, A. E. Eaker, D. S. Stoffer, A. Bell, R. Zarnegar, and M. C. DeFrances. 2007. PI3K is negatively regulated by PIK3IP1, a novel p110 interacting protein. Biochemical and biophysical research communications 358:66-72.
2. Fruman, D. A., and G. Bismuth. 2009. Fine tuning the immune response with PI3K. Immunological reviews 228:253-272.
3. Engelman, J. A., J. Luo, and L. C. Cantley. 2006. The evolution of phosphatidylinositol 3-kinases as regulators of growth and metabolism. Nat Rev Genet 7:606-619.
4. Okkenhaug, K., and B. Vanhaesebroeck. 2003. PI3K in lymphocyte development, differentiation and activation. Nature reviews. Immunology 3:317-330.
5. Altomare, D. A., and J. R. Testa. 2005. Perturbations of the AKT signaling pathway in human cancer. Oncogene 24:7455-7464.
6. Luo, J., B. D. Manning, and L. C. Cantley. 2003. Targeting the PI3K-Akt pathway in human cancer: rationale and promise. Cancer cell 4:257-262.
7. Wong, K. K., J. A. Engelman, and L. C. Cantley. 2009. Targeting the PI3K signaling pathway in cancer. Curr Opin Genet Dev 20:87-90.
8. Gewinner, C., Z. C. Wang, A. Richardson, J. Teruya-Feldstein, D. Etemadmoghadam, D. Bowtell, J. Barretina, W. M. Lin, L. Rameh, L. Salmena, P. P. Pandolfi, and L. C. Cantley. 2009. Evidence that inositol polyphosphate 4-phosphatase type II is a tumor suppressor that inhibits PI3K signaling. Cancer cell 16:115-125.
9. Seminario, M. C., and R. L. Wange. 2003. Lipid phosphatases in the regulation of T cell activation: living up to their PTEN-tial. Immunological reviews 192:80-97.
10. Manning, B. D. 2004. Balancing Akt with S6K: implications for both metabolic diseases and tumorigenesis. The Journal of cell biology 167:399-403.
11. Manning, B. D., and L. C. Cantley. 2007. AKT/PKB signaling: navigating downstream. Cell 129:1261-1274.
12. Zoncu, R., A. Efeyan, and D. M. Sabatini. 2011. mTOR: from growth signal integration to cancer, diabetes and ageing. Nat Rev Mol Cell Biol 12:21-35.
13. Kashiwada, M., P. Lu, and P. B. Rothman. 2007. PIP3 pathway in regulatory T cells and autoimmunity. Immunologic research 39:194-224.
14. Sauer, S., L. Bruno, A. Hertweck, D. Finlay, M. Leleu, M. Spivakov, Z. A. Knight, B. S. Cobb, D. Cantrell, E. O'Connor, K. M. Shokat, A. G. Fisher, and M. Merkenschlager. 2008. T cell receptor signaling controls Foxp3 expression via PI3K, Akt, and mTOR. Proceedings of the National Academy of Sciences of the United States of America 105:7797-7802.
15. Crellin, N. K., R. V. Garcia, and M. K. Levings. 2007. Altered activation of AKT is required for the suppressive function of human CD4+CD25+T regulatory cells. Blood 109:2014-2022.
16. He, X., Z. Zhu, C. Johnson, J. Stoops, A. E. Eaker, W. Bowen, and M. C. DeFrances. 2008. PIK3IP1, a negative regulator of PI3K, suppresses the development of hepatocellular carcinoma. Cancer research 68:5591-5598.
17. Gilbert, D. C., A. McIntyre, B. Summersgill, E. Missiaglia, N. C. Goddard, I. Chandler, R. A. Huddart, and J. Shipley. 2010. Minimum regions of genomic imbalance in stage I testicular embryonal carcinoma and association of 22q loss with relapse. Genes Chromosomes Cancer 50:186-195.
18. Crabtree, G. R., and K. Corbit. Use of CRKD as a breast cancer marker and cancer therapy target. U.S. patent application Ser. No. 11/191,457. Publication No. 20060110395A1. May 25, 2006.
19. Fayard, E., G. Moncayo, B. A. Hemmings, and G. A. Hollander. 2010. Phosphatidylinositol 3-kinase signaling in thymocytes: the need for stringent control. Science signaling 3:re5.
20. DeFrances, M. C., D. R. Debelius, J. Cheng, and L. P. Kane. 2012. Inhibition of T-cell activation by PIK3IP1. European journal of immunology 42:2754-2759.
21. Liao, X. C., and D. R. Littman. 1995. Altered T cell receptor signaling and disrupted T cell development in mice lacking Itk. Immunity 3:757-769.
22. Liu, K. Q., S. C. Bunnell, C. B. Gurniak, and L. J. Berg. 1998. T cell receptor-initiated calcium release is uncoupled from capacitative calcium entry in Itk-deficient T cells. Journal of Experimental Medicine 187:1721-1727.
23. Miller, A. T., H. M. Wilcox, Z. Lai, and L. J. Berg. 2004. Signaling through Itk promotes T helper 2 differentiation via negative regulation of T-bet. Immunity 21:67-80.
24. Yin, C. C., O. H. Cho, K. E. Sylvia, K. Narayan, A. L. Prince, J. W. Evans, J. Kang, and L. J. Berg. 2013. The Tec kinase ITK regulates thymic expansion, emigration, and maturation of gammadelta NKT cells. Journal of immunology 190:2659-2669.
25. Prince, A. L., Z. Kraus, S. A. Carty, C. Ng, C. C. Yin, M. S. Jordan, P. L. Schwartzberg, and L. J. Berg. 2014. Development of innate CD4+ and CD8+ T cells in Itk-deficient mice is regulated by distinct pathways. Journal of immunology 193:688-699.
26. Prince, A. L., L. B. Watkin, C. C. Yin, L. K. Selin, J. Kang, P. L. Schwartzberg, and L. J. Berg. 2014. Innate PLZF+CD4+ alphabeta T cells develop and expand in the absence of Itk. Journal of immunology 193:673-687.
27. Tubo, N. J., and M. K. Jenkins. 2014. TCR signal quantity and quality in CD4 T cell differentiation. Trends in immunology 35:591-596.
28. Kim, M. S., M. Radinger, and A. M. Gilfillan. 2008. The multiple roles of phosphoinositide 3-kinase in mast cell biology. Trends in immunology 29:493-501.

### SEQUENCES

(1) Mouse PIK3IP1 Reference sequence of original
clone (obtained from Origene)
Mouse PIK3IP1 GenBank #BC055920.1
PIK3IP1 is a 264 amino acid sequence.
28567.3732 Daltons (predicted)
Signal peptide is 20 amino acids long
Kringle domain is 74 amino acids long
Transmembrane domain is 26 amino acids long
p85 domain is 21 amino acids long
Mouse PIK3IP1
(SEQ ID NO: 1)
M L L A W V H T F L L S N M L L A E A Y G S G G
C F W D N G H L Y R E D Q P S P A P G L R C L N
W L A A Q G S R E S L T E P S P G N H N Y C R N
P D Q D P R G P W C Y I S S E T G V P E K R P C
E D V S C P E T T S Q A P P P S S A M E L E E K
S G A P G D K E A Q V F P P A N A L P A R S E A
A E V Q P V I G I S Q L V R M N S K E K K D L G
T L G Y V L G I T M M V I I L A I G A G I I V G
Y T Y K R G K D L K E Q H E K K A C E R E M Q R
I T L P L S A F T N P T C E T V D E N T I I V H
S N Q T P A D V Q E G S T L L T G Q A G T P G A
Kringle domain from mouse PIK3IP1
(SEQ ID NO: 2)
G G C F W D N G H L Y R E D Q P S P A P G L R C
L N W L A A Q G S R E S L T E P S P G N H N Y C
R N P D Q D P R G P W C Y I S S E T G V P E K R
P C E D V S C P E T
Transmembrane domain from mouse PIK3IP1
(SEQ ID NO: 3)
L G T L G Y V L G I T M M V I I L A I G A G I I
V G
p85-like domain from mouse PIK3IP1
(SEQ ID NO: 4)
G K D L K E Q H E K K A C E R E M Q R I T L P
(2) Flag tagged wt-PIK3IP1 (mouse) Translation:
Flag tagged wt-PIK3IP1 (mouse)
(SEQ ID NO: 5)
M A L P V T A L L L P L A L L L H A A R P D Y K
D D D D K I D G G C F W D N G H L Y R E D Q P S
P A P G L R C L N W L A A Q G S R E S L T E P S
P G N H N Y C R N P D Q D P R G P W C Y I S S E
T G V P E K R P C E D V S C P E T T S Q A P P P
S S A M E L E E K S G A P G D K E A Q V F P P A
N A L P A R S E A A E V Q P V I G I S Q L V R M
N S K E K K D L G T L G Y V L G I T M M V I I L
A I G A G I I V G Y T Y K R G K D L K E Q H E K
K A C E R E M Q R I T L P L S A F T N P T C E T
V D E N T I I V H S N Q T P A D V Q E G S T L L
T G Q A G T P G A
hCD8 signal sequence from flag tagged
wt-PIK3IP1 (mouse)
(SEQ ID NO: 6)
M A L P V T A L L L P L A L L L H A A R P
Flag epitope tag from flag tagged wt-PIK3IP1
(mouse)
(SEQ ID NO: 7)
D Y K D D D D K I D
Kringle domain from flag tagged wt-PIK3IP1
(mouse)
(SEQ ID NO: 8)
G G C F W D N G H L Y R E D Q P S P A P G L R C
L N W L A A Q G S R E S L T E P S P G N H N Y C
R N P D Q D P R G P W C Y I S S E T G V P E K R
P C E D V S C P E T
Transmembrane domain from flag tagged
wt-PIK3IP1 (mouse)
(SEQ ID NO: 9)
L G T L G Y V L G I T M M V I I L A I G A G I I
V G
p85-like domain from flag tagged wt-PIK3IP1
(mouse)
(SEQ ID NO: 10)
G K D L K E Q H E K K A C E R E M Q R I T L P
(P E T) remaining kringle residues in kringle
deletion mutant (which does include the CD8
signal sequence and Flag tag)
(R)-last residue of cytoplasmic truncation mutant
(3) hCD8 ecto/tm-- PIK3IP1 cyto chimera
(SEQ ID NO: 11)
M A L P V T A L L L P L A L L L H A A R P S Q F
R V S P L D R T W N L G E T V E L K C Q V L L S
N P T S G C S W L F Q P R G A A A S P T F L L Y
L S Q N K P K A A E G L D T Q R F S G K R L G D
T F V L T L S D F R R E N E G Y Y F C S A L S N
S I M Y F S H F V P V F L P A K P T T T P A P R
P P T P A P T I A S Q P L S L R P E A C R P A A
G G A V H T R G L D F A C D I Y I W A P L A G T
C G V L L L S L V I T L Y C N H R N R R R S Y T
Y K R G K D L K E Q H E K K A C E R E M Q R I T
L P L S A F T N P T C E T V D E N T I I V H S N
Q T P A D V Q E G S T L L T G Q A G T P G A
(4) Amino acid sequence of human PIK3IP1 (NCBI
reference NP_443112)
(SEQ ID NO: 12)
M L L A W V Q A F L V S N M L L A E A Y G S G G
C F W D N G H L Y R E D Q T S P A P G L R C L N
W L D A Q S G L A S A P V S G A G N H S Y C R N
P D E D P R G P W C Y V S G E A G V P E K R P C
E D L R C P E T T S Q A L P A F T T E I Q E A S
E G P G A D E V Q V F A P A N A L P A R S E A A
A V Q P V I G I S Q R V R M N S K E K K D L G T
L G Y V L G I T M M V I I I A I G A G I I L G Y
S Y K R G K D L K E Q H D Q K V C E R E M Q R I
T L P L S A F T N P T C E I V D E K T V V V H T
S Q T P V D P Q E G S T P L M G Q A G T P G A
Extracellular domain from human PIK3IP1
(SEQ ID NO: 13)
M L L A W V Q A F L V S N M L L A E A Y G S G G
C F W D N G H L Y R E D Q T S P A P G L R C L N
W L D A Q S G L A S A P V S G A G N H S Y C R N
P D E D P R G P W C Y V S G E A G V P E K R P C
E D L R C P E T T S Q A L P A F T T E I Q E A S
E G P G A D E V Q V F A P A N A L P A R S E A A
A V Q P V I G I S Q R V R M N S K E K K D
Kringle Domain from human PIK3IP1
(SEQ ID NO: 14)
G G C F W D N G H L Y R E D Q T S P A P G L R C
L N W L D A Q S G L A S A P V S G A G N H S Y C
R N P D E D P R G P W C Y V S G E A G V P E K R
P C E D L R C P E T

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

We claim:

1. An isolated antibody or antigen-binding fragment thereof that specifically binds to a PIK3IP1 polypeptide, wherein the antibody or antigen-binding fragment thereof reduces the activity of T cells involved in mediating an inflammatory disease.

2. The antibody of claim 1, wherein the antibody binds to an extracellular domain of PIK3IP1.

3. The antibody of claim 1, wherein the antibody binds to the amino acid sequence SEQ ID NO:13.

4. The antibody of claim 1, wherein the antibody binds to a kringle domain of PIK3IP1.

5. The antibody of claim 1, wherein the antibody binds to the amino acid sequence SEQ ID NO:14.

6. The antibody of claim 1, wherein the antibody is a monoclonal antibody.

7. An isolated antibody or antigen-binding fragment thereof that specifically binds to a PIK3IP1 polypeptide, wherein the antibody or antigen-binding fragment thereof reduces the activity of mast cells involved in mediating an allergic condition or disease.

8. The antibody of claim 7, wherein the antibody binds to an extracellular domain of PIK3IP1.

9. The antibody of claim 7, wherein the antibody binds to the amino acid sequence SEQ ID NO:13.

10. The antibody of claim 7, wherein the antibody binds to a kringle domain of PIK3IP1.

11. The antibody of claim 7, wherein the antibody binds to the amino acid sequence SEQ ID NO:14.

12. The antibody of claim 7, wherein the antibody is a monoclonal antibody.

13. A method for treating or preventing an inflammatory disease mediated by T cells, comprising administering to a subject in need thereof an effective amount of the antibody or antigen-binding fragment thereof of claim 1.

14. The method of claim 13, wherein the antibody binds to an extracellular domain of PIK3IP1.

15. The method of claim 13, wherein the antibody binds to the amino acid sequence SEQ ID NO:13.

16. The method of claim 13, wherein the antibody binds to a kringle domain of PIK3IP1.

17. The method of claim 13, wherein the antibody binds to the amino acid sequence SEQ ID NO:14.

18. The method of claim 13, wherein the antibody is a monoclonal antibody.

19. The method of claim 13, wherein the inflammatory disease is selected from diabetes mellitus, multiple sclerosis, systemic lupus erythematosus, rheumatoid arthritis, psoriatic arthritis, or psoriasis.

20. A method for treating or preventing an allergic condition or disease mediated by mast cells, comprising administering to a subject in need thereof an effective amount of the antibody or antigen-binding fragment thereof of claim 7.
